SEQUENCE LISTING (12 sequences)

SEQ ID NO: 1 - DNA, 993 bp, Saccharomyces cerevisiae
gatttttataatgacgaaaaaaaaaaaattggaaagaaaaagcttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaaggaattcgagctaagcggccgc

SEQ ID NO: 2 - DNA, 816 bp, Saccharomyces cerevisiae
ccatatgatcatgtgtcgtcgcacacatatatatatgcctgtatgtgtcagcactaaagttgcctggccatccacgctatatatacacgcctggcggatctgctcgaggattgcctacgcgtgggcttgatccaccaaccaacgctcgccaaatgaactggcgctttggtcttctgccatcgtccgtaaaccccggccaaagagaccggaaagatcggtgaaaacatcttgatcttgctcccgggaattttagattcaggtaggaaattgattacatcaatactgttaccctgaatcatattcgacgatgtcgtctcacacggaaatataattcatttcttggttttccaaaaaaattttcattttttttcacttttttgtttcgtcctcctttttttttttttttttttattttttttcctgtgttcacctttttttttttcagttgacatctttctgcattcttttctgtgtttttttttttttttttcgtttttccattgttcgttcgttgcctgttttttcgccctattgttctcgagcctaaaaattttttcctttcctgctttcctttcttcgttcaaagtttcctattccattgttctctttggtaaactcattgttgtcggaactcagatatattcaggtcaatttactgtacttcaattgacttttttcttgaaatttcaacttgccttttcaacttgttcttcttttttaatcttattctacactttagttcccttaccttgttcctaattattgtctagcaaaaagaaaacatacacctatttcattcacacactgcagaaaatg

SEQ ID NO: 3 - protein, 500 aa, Saccharomyces cerevisiae
  MISC_FEATURE: S. cerevisiae ALD6 (Accession NO. NP_015264)
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 4 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 5 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 6 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 7 - protein, 250 aa, Saccharomyces cerevisiae
MPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 8 - protein, 669 aa, Saccharomyces cerevisiae
  MISC_FEATURE: S. cerevisiae FPS1 (Accession No. NP_013057)
MSNPQKALNDFLSSESVHTHDSSRKQSNKQSSDEGRSSSQPSHHHSGGTNNNNNNNNNNNNSNNNNNGNDGGNDDDYDYEMQDYRPSPQSARPTPTYVPQYSVESGTAFPIQEVIPSAYINTQDINHKDNGPPSASSNRAFRPRGQTTVSANVLNIEDFYKNADDAHTIPESHLSRRRSRSRATSNAGHSANTGATNGRTTGAQTNMESNESPRNVPIMVKPKTLYQNPQTPTVLPSTYHPINKWSSVKNTYLKEFLAEFMGTMVMIIFGSAVVCQVNVAGKIQQDNFNVALDNLNVTGSSAETIDAMKSLTSLVSSVAGGTFDDVALGWAAAVVMGYFCAGGSAISGAHLNPSITLANLVYRGFPLKKVPYYFAGQLIGAFTGALILFIWYKRVLQEAYSDWWMNESVAGMFCVFPKPYLSSGRQFFSEFLCGAMLQAGTFALTDPYTCLSSDVFPLMMFILIFIINASMAYQTGTAMNLARDLGPRLALYAVGFDHKMLWVHHHHFFWVPMVGPFIGALMGGLVYDVCIYQGHESPVNWSLPVYKEMIMRAWFRRPGWKKRNRARRTSDLSDFSYNNDDDEEFGERMALQKTKTKSSISDNENEAGEKKVQFKSVQRGKRTFGGIPTILEEEDSIETASLGATTTDSIGLSDTSSEDSHYGNAKKVT

SEQ ID NO: 9 - protein, 348 aa, Saccharomyces cerevisiae
  MISC_FEATURE: S. cerevisiae ADH1 (Accession No. NP_014555)
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 10 - protein, 386 aa, Saccharomyces cerevisiae
MSAATVGKPIKCIAAVAYDAKKPLSVEEITVDAPKAHEVRIKIEYTAVCHTDAYTLSGSDPEGLFPCVLGHEGAGIVESVGDDVITVKPGDHVIALYTAECGKCKFCTSGKTNLCGAVRATQGKGVMPDGTTRFHNAKGEDIYHFMGCSTFSEYTVVADVSVVAIDPKAPLDAACLLGCGVTTGFGAALKTANVQKGDTVAVFGCGTVGLSVIQGAKLRGASKIIAIDINNKKKQYCSQFGATDFVNPKEDLAKDQTIVEKLIEMTDGGLDFTFDCTGNTKIMRDALEACHKGWGQSIIIGVAAAGEEISTRPFQLVTGRVWKGSAFGGIKGRSEMGGLIKDYQKGALKVEEFITHRRPFKEINQAFEDLHNGDCLRTVLKSDEIK

SEQ ID NO: 11 - protein, 678 aa, Saccharomyces cerevisiae
MGLLALGTPLQWFESRTYNEHIRDEGIEQLLYIFQAAGKRDNDPLFWGDELEYMVVDFDDKERNSMLDVCHDKILTELNMEDSSLCEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 12 - protein, 428 aa, Saccharomyces cerevisiae
MTAAKPNPYAAKPGDYLSNVNNFQLIDSTLREGEQFANAFFDTEKKIEIARALDDFGVDYIELTSPVASEQSRKDCEAICKLGLKAKILTHIRCHMDDAKVAVETGVDGVDVVIGTSKFLRQYSHGKDMNYIAKSAVEVIEFVKSKGIEIRFSSEDSFRSDLVDLLNIYKTVDKIGVNRVGIADTVGCANPRQVYELIRTLKSVVSCDIECHFHNDTGCAIANAYTALEGGARLIDVSVLGIGERNGITPLGGLMARMIVAAPDYVKSKYKLHKIRDIENLVADAVEVNIPFNNPITGFCAFTHKAGIHAKAILANPSTYEILDPHDFGMKRYIHFANRLTGWNAIKARVDQLNLNLTDDQIKEVTAKIKKLGDVRSLNIDDVDSIIKNFHAEVSTPQVLSAKKNKKNDSDVPELATIPAAKRTKPSA

(19) United States
(12) Patent Application Publication
(10) Pub. No.: US 2018/0030482 A1
(43) Pub. Date: Feb. 1, 2018

(54) Use of acetaldehyde in the fermentative production of ethanol

(71) Applicant: Moralco B.V., Ede (NL)

(72) Inventor: Johannes Adrianus Maria de Bont, Wageningen (NL)

(73) Assignee: Moralco B.V., Ede (NL)

(21) Appl. No.: 15/304,874

(22) PCT Filed: Apr. 16, 2015
(86) PCT No.: PCT/NL2015/050256

§ 371 Date: Oct. 18, 2016

(30) Foreign Application Priority Data
Apr. 18, 2014 (EP) 14165291.7
Jun. 6, 2014 (EP) 14171443.6
Nov. 26, 2014 (EP) 14194989.1

(51) Int. Cl. C12P 7/06; C12G 3/12; C12F 3/06

(52) U.S. Cl. CPC C12P 7/06; C12F 3/06; C12G 3/12

(57) ABSTRACT

The instant invention relates to processes and systems for the fermentative production of ethanol. The ethanol is produced by fermenting a fermentable carbohydrate with a yeast, whereby acetaldehyde is externally supplied to the yeast cell for reducing glycerol by-product formation by the yeast cell, for improving the performance of yeast at high ethanol levels and/or for suppression of infections during the fermentation. The acetaldehyde that is externally supplied to the fermentation medium can be produced by catalytic oxidation of ethanol. Advantageously, acetaldehyde production from ethanol is integrated in a system for ethanol production. Thus, in another aspect the invention relates to a system for producing ethanol, e.g. an ethanol plant, which system, in addition to the usual means for fermentative production of ethanol, comprises a means for producing acetaldehyde by catalytic oxidation of ethanol. The invention further relates to a process for disinfecting fermentation equipment such as fermenters and bioreactors, as well as fermentations feedstocks, wherein the equipment and/or feedstocks is disinfected with high concentrations of acetaldehyde, which are then diluted to non-toxic concentrations by addition of the fermentation medium.

### FIELD OF THE INVENTION

The present invention relates to the fermentative production of ethanol. In particular the invention relates to processes, as well as production facilities, wherein yeasts ferment a source of carbohydrate to ethanol and wherein acetaldehyde is used for one or more of 1) reduction of glycerol by-product formation; 2) reduction of inhibition of fermentation at high ethanol concentrations, and 3) disinfection of the fermenter prior to fermentation.

### BACKGROUND OF THE INVENTION

Ethanol is an important liquid energy carrier that is produced worldwide from renewable feedstocks. Sugars from biomass are converted in biological processes that usually are based on the yeast Saccharomyces cerevisiae. The sugars used as first generation feedstocks are glucose and fructose as obtained from starch or sucrose from crops such as wheat, corn and sugar cane. Alternatively, ethanol can be produced from second generation feedstocks including glucose from cellulose and xylose from hemicellulose. Bagasse and non-starch parts of the corn and wheat plants are considered as good sources for second generation processes.

Large-scale production facilities for ethanol were already in operation at the end of the 19^{th} century and in the 20^{th} century many production plants were operated. Currently, ethanol is produced worldwide at large scales.

In view of the long-standing operation of ethanol-production plants, it might be expected that optimal procedures in terms of economics have been reached in converting sugars into ethanol. Surprisingly, still to date, a number of issues remain that are open for improvements that will lead to better yields of ethanol from sugar. At least three of such issues that continue to present themselves as having a negative impact on overall process yield include: 1) contaminations by bacteria and by wild yeasts; 2) production of glycerol as unwanted byproduct; and, 3) inhibition of the fermentation at high ethanol concentrations.

An overview of contaminations in fuel ethanol fermentations has been given by Narendranath (The Alcohol Textbook, 4^{th} edition, editors K. A. Jacques, T. P. Lyons and D. R Kensall, pages 287-297, 2003), who emphasizes that bacterial contaminations are a major cause of reduction in ethanol yield during fermentations by S. cerevisiae. Sugar consumed by bacteria not only divert it away from ethanol production and hence yields for ethanol are reduced but also the sugar is converted into acids such as lactic and acetic acids which are inhibitory to yeast metabolism. Bacterial contaminations mainly are lactic acid bacteria because they are able to cope under anoxic conditions with low pH-values in combination with relatively high ethanol concentrations. These conditions prevail during the S. cerevisiae fermentations. Infecting microbes during large scale fermentations are difficult to combat and they may show up unexpectedly. A number of methodologies (antibiotics, chlorine dioxide) are available in fighting contaminants but they have drawbacks. Antibiotics increase costs and they are undesirable in view of potential risks with bacterial resistance. The use of chlorine dioxide involves both cost and environmental aspect.

Glycerol is concomitantly produced during anoxic ethanol production by S. cerevisiae. The events leading to and controlling the glycerol formation in yeast have been investigated and described in detail amongst others by Van Dijken and Scheffers (1986, FEMS Microbiol. Rev., 32:199-224). They highlighted the relation between biomass and an excess of NADH resulting from its formation.

Under strictly anoxic growth conditions, substrate level phosphorylation in glycolysis is the sole source of ATP in S. cerevisiae and enables a net yield of 2 ATP for each molecule of glucose converted to two molecules of pyruvate. Dissimilation of glucose to ethanol via glycolysis and pyruvate decarboxylation is NAD(H) neutral. But in the production of yeast biomass, a net formation of NADH from NAD occurs. The yeast keeps its redox balance neutral by producing glycerol. The amount of glycerol produced relative to the amount of ethanol produced varies and it depends on at least on two factors: 1) the amount of biomass produced relative to the amount of ethanol produced, and 2) glycerol can also be necessary as a compatible solute to allow the yeast to counteract osmotic stresses as they occur during fermentation.

Several metabolic engineering approaches have been undertaken to reduce quantities of glycerol produced during alcoholic fermentations and thus optimize ethanol production. Nissen et al. (2000, Metabol. Engineer. 2:69-77) have concentrated on engineering the ammonium assimilation route. Others have concentrated on genetic modifications of the levels of NAD-dependent glycerol-3-phosphate dehydrogenases (Hubman et al. 2011, Appl. Environ. Microbiol. 77:5857-5867), glycerol-3-phosphatases (Granath et al. 2005, Yeast 22:1257-1268) and the Fps1p plasma membrane channel for glycerol.

An acetylating acetaldehyde dehydrogenase activity (EC 1.2.1.10) has been expressed heterologously in S. cerevisiae. (Guadeloupe-Medina et al. 2010, Appl. Environ. Microbiol. 76:190-195). By having acetic acid in the external environment, the glycerol production can be reduced since acetyl-CoA can be reduced by the newly acquired enzymatic reaction. In another approach, by using CO₂ as electron acceptor for the reoxidation of NADH and by functional expressing ribulose-1,5-bisphosphate carboxylase an enhanced production of ethanol was obtained (Guadeloupe-Medina et al. 2013, Biotechnol. Biofuels 6:125). Again a different approach was by deleting an NAD⁺-dependent glycerol-3-phosphate dehydrogenase and by simultaneously expressing a non-phosphorylating NADP⁺-dependent glyceraldehyde-3-phosphate dehydrogenase from Bacillus cereus (Guo et al. 2011, Metabolic Engineering 13:49-59).

The implications of genetically modifying the glycerol metabolism in terms of robustness of the ethanol-producing strains has been studied (Pagliardini et al. 2013, Microbial Cell Factories 12:29). The reduction of the glycerol yield also led to severe reduction in the fermentation kinetics, in cell viability, and in the final ethanol concentration reached. Despite such challenges, a genetically modified yeast, improved for ethanol fermentation has been obtained that functions in an industrial setting. This strain, TransFerm™, is commercialized jointly by Lallemand Biofuels & Distilled Spirits and Mascoma and produces about 30% less glycerol as compared to conventional yeasts whereas ethanol production is reported to increase in the order of 4%.

At higher levels of ethanol in the fermentation broth, the production of ethanol slows down and eventually comes to an halt. The inhibitory role of ethanol on S. cerevisiae is not fully understood although many mechanisms have been reported in this respect (Ding et al. 2009. Appl. Microbiol. Biotechnol. 85:253-263).

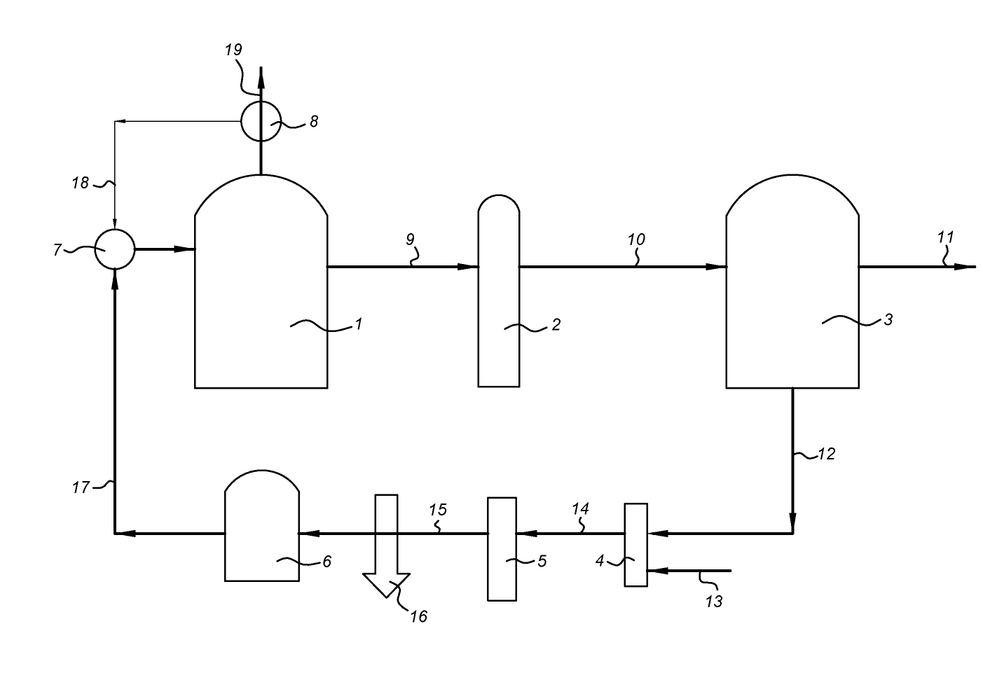

Barber et al. (2002, Biotechnol. Lett. 24: 891-895) disclose the ability of low levels of acetaldehyde to increase the specific growth rate of ethanol-stressed cultures of S. cerevisiae. However, FIG. 2 of Barber et al. shows that the acetaldehyde is not fed into the medium until after the cells have stopped growing and entered stationary phase. Similarly, Roustan and Sablayrolles (2002, J. BioSci. Bioeng. 93:367-375) disclose the addition of low concentrations of acetaldehyde to alcoholic yeast fermentations that are well into stationary phase.

Vriesekoop et al. (2007, Biotechnol. Lett. 29:1099-1103) disclose that addition of acetaldehyde to ethanol-stressed S. cerevisiae stimulates growth and glycolysis and rectifies an ethanol-induced redox imbalance. In a follow-up study Vriesekoop et al. (2009, FEMS Yeast Res, 9: 365-371) disclose that acetaldehyde does not appear to be a universal ameliorating agent for yeasts exposed to ethanol stress when tested among a wide range of different yeast species. In these studies the yeasts are transferred into media already containing stress-inducing concentrations of ethanol rather than that the ethanol is produced by fermentation of a carbohydrate source.

None of these publications disclose the addition of acetaldehyde to growing alcoholic yeast fermentations and/or before high ethanol concentrations are reached and at a rate sufficient to prevent or reduce glycerol byproduct formation.

It is an object of the present invention to provide for means and methods for improving the fermentative production of ethanol. Amongst others, it is an object to provide for means and methods that address the above-mentioned issues that continue to present themselves as having a negative impact on overall process yield, thereby enhancing the overall economics of the ethanol processes at large production scales.

### SUMMARY OF THE INVENTION

In a first aspect the invention relates to a process for producing ethanol comprising: a) fermenting a medium with a yeast cell in a fermenter, whereby the medium contains or is fed with: i) a source of a fermentable carbohydrate; and, ii) a source of acetaldehyde; and whereby the yeast cell ferments the fermentable carbohydrate and the acetaldehyde to ethanol; and, b) recovery of the ethanol from the medium, wherein preferably, the acetaldehyde is present in or fed into the medium at least during a stage in the process when the growth rate of the yeast cell is at least 0.005 h⁻¹, and/or wherein preferably, the acetaldehyde is present in or fed into the medium at least prior to the ethanol in the medium reaching a concentration that is higher than 50 kg/m³.

A preferred process according to the invention, is a process wherein: a) in a first phase of the process before a threshold ethanol concentration in the medium is reached, the rate of the acetaldehyde fed into the medium is controlled to maintain an acetaldehyde concentration of at least 0.0009 kg/m³ and, preferably no more than 1.0 kg/m³; and, b) in a second phase of the process after the threshold ethanol concentration in the medium is reached, the rate of the acetaldehyde fed into the medium is controlled to maintain an acetaldehyde concentration of no more than 0.3 kg/m³, and preferably at least 0.0009 kg/m³, and wherein the threshold ethanol concentration is between 40 and 100 kg/m³. Preferably, in the process the acetaldehyde concentration in the medium is monitored on-line in an off-gas stream from the fermenter, preferably using a mass spectrometer or a gas chromatograph.

In a process according to the invention, the acetaldehyde is preferably fed into the medium in a liquid form or in gaseous form, wherein more preferably the acetaldehyde in gaseous form is mixed with at least a part of the off-gas stream from the fermenter that is recycled back into the fermenter.

In a second aspect the invention relates to a the yeast cell for use in a process of the invention. Preferably the yeast cell is of a genus selected from the group consisting of Saccharomyces, Kazachstania and Naumovia, wherein preferably the yeast cell belongs to a species selected from the group consisting of Saccharomyces cerevisiae, S. bayanus, S. bulderi, S. cervazzii, S. cariocanus, S. castellii, S. dairenensis, S. exiguus, S. kluyveri, S. kudriazevii, S. mikatae, S. paradoxus, S. pastorianus, S. turicensis, and S. unisporus. Preferably, the yeast cell has one or more modifications selected from the group consisting of: a) a genetic modification that increases resistance to acetaldehyde as compared to a corresponding unmodified parent strain, whereby preferably the cell with increased resistance to acetaldehyde is obtained by one or more of: i) evolutionary engineering; ii) a genetic modification that increases specific NADH-dependent alcohol dehydrogenase activity, whereby preferably the alcohol dehydrogenase has an amino acid sequence with at least 70% sequence identity to SEQ ID NO: 9; iii) a genetic modification that increases the specific NADH-dependent alcohol dehydrogenase and glutathione-dependent aldehyde dehydrogenase activities, whereby preferably the bifunctional NADH-dependent alcohol dehydrogenase and glutathione-dependent aldehyde dehydrogenase has an amino acid sequence with at least 70% sequence identity to SEQ ID NO: 10; iv) a genetic modification that increases the intracellular glutathione level in the cell, whereby preferably, the genetic modification comprises at least the overexpression of a gene encoding a γ-glutamylcysteine synthetase, whereby preferably the γ-glutamylcysteine synthetase has an amino acid sequence with at least 70% sequence identity to SEQ ID NO: 11; and, v) a genetic modification that increases the intracellular lysine level in the cell, whereby preferably the genetic modification confers resistance to S-2-aminoethyl-L-cysteine, or the genetic modification comprises reducing or eliminating the expression of a gene encoding a amino acid sequence with at least 70% sequence identity to SEQ ID NO: 12; b) a genetic modification that reduces or eliminates endogenous aldehyde dehydrogenase activity, whereby preferably the genetic modification reduces or eliminates expression of endogenous S. cerevisiae ALD6 gene or an orthologue thereof; c) a genetic modification that reduces or eliminates NADH-dependent glycerol synthesis, whereby preferably the genetic modification is a modification that reduces or eliminates the expression of one or more of the S. cerevisiae GPD1, GPD2, HOR2 and RHR2 genes or orthologues thereof; d) a genetic modification that reduces or eliminates transport of glycerol, whereby preferably the genetic modification is a modification that reduces or eliminates the expression of the S. cerevisiae FPS1 gene or an orthologue thereof; and, e) a genetic modification that introduces into the cell at least one of: i) expression of an exogenous xylose isomerase gene, which gene confers to the cell the ability to isomerize xylose into xylulose; and, ii) expression of exogenous genes coding for a L-arabinose isomerase, a L-ribulokinase and a L-ribulose-5-phosphate 4-epimerase, which genes together confer to the cell the ability to convert L-arabinose into D-xylulose 5-phosphate, whereby the cell further preferably comprises genetic modifications that increase the specific activities of one or more of xylulose kinase, ribulose-5-phosphate isomerase, ribulose-5-phosphate 3-epimerase, transketolase and transaldolase; and a genetic modification that reduces or eliminates unspecific aldose reductase activity.

In a third aspect, the invention pertains to a process for disinfecting a fermenter, wherein the process comprises the steps of: i) supplying to the fermenter an amount of acetaldehyde resulting in a concentration of acetaldehyde of at least 1 kg/m³, (and incubating the acetaldehyde in the fermenter for at least 5 minutes), whereby the amount of acetaldehyde supplied is such that upon supply of the medium and the yeast cell to the fermenter, the concentration of acetaldehyde is diluted to no more than 2.0 kg/m³; and, ii) supplying medium and optionally yeast cells to the fermenter in an amount to dilute the acetaldehyde to a concentration of no more than 2.0 kg/m³; whereby, preferably, step i) the acetaldehyde is supplied into the fermenter in gas phase and/or the acetaldehyde is brought into the gas phase and or kept in the gas phase in the fermenter.

In the processes of the invention, the acetaldehyde can be produced by catalytic oxidation of ethanol, preferably using a catalyst comprising one or more of a noble metal, an alloy thereof and oxides thereof, in the presence of oxygen, wherein preferably the noble metal is selected from silver, copper, platinum and gold. In a preferred embodiment, the acetaldehyde is produced by catalytic oxidation of a part of the ethanol obtained in a process of the invention for producing ethanol, whereby preferably, the acetaldehyde is produced at a site in the vicinity of the site where the ethanol is produced.

In a fourth aspect, the invention pertains to a system for producing ethanol in a process according to any one of the preceding claims, wherein the system comprises a means for fermentation of a medium to an ethanol-containing beer, a means for distillation for recovery of ethanol from the beer and a means for supplying acetaldehyde to the medium, wherein, preferably, the system further comprises a means for producing acetaldehyde by catalytic oxidation of ethanol, such as a reactor holding a catalyst as defined hereinabove. The system preferably is a system wherein: a) the system is configured to produce acetaldehyde by catalytic oxidation from a part of the ethanol obtained from the means for distillation, optionally after storage of the ethanol; and, b) optionally, the system is configured to supply the acetaldehyde produced in a) to the medium, optionally after storage of the acetaldehyde. In one embodiment, the system comprises a means for monitoring the acetaldehyde concentration and optionally the ethanol concentration, in the fermentation medium and a means for controlling the rate of the acetaldehyde supply into the medium in the fermenter, wherein preferably, the means for controlling the rate of acetaldehyde supply into the medium receives input from the means for monitoring the acetaldehyde concentration, and optionally the ethanol concentration, to maintain an acetaldehyde concentration in the medium in accordance with a process of the invention, wherein preferably, the means for controlling the rate of acetaldehyde supply into the medium further receives input from the means for monitoring the ethanol concentration in the medium to further control the acetaldehyde concentration in the medium as a function of the ethanol concentration in accordance with a process of the invention. Preferably, in the system the means for controlling the rate of acetaldehyde supply into the medium receives input from the means for monitoring the acetaldehyde concentration to control an acetaldehyde concentration in the medium in accordance with a process of the invention, wherein preferably, the means for controlling the rate of acetaldehyde supply into the medium further receives input from the means for monitoring the ethanol concentration in the medium to further control the acetaldehyde concentration in the medium as a function of the ethanol concentration in accordance with a process of the invention.

In a fifth aspect, the invention relates to the use of acetaldehyde in a yeast fermentation process for producing ethanol, wherein the acetaldehyde is used for at least one of: a) reducing the formation of glycerol; b) improving the performance of the yeast at high ethanol concentration; and, c) suppression of infection during the fermentation process, wherein, preferably the process is a process according to the invention.

In a sixth aspect, the invention relates to the use acetaldehyde for disinfecting a fermenter and/or a feedstock for a fermentation process, wherein, preferably the fermenter is disinfected in a process according to the invention.

### DESCRIPTION OF THE INVENTION

Definitions

Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2. 10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred. Alternatively percentage similarity or identity may be determined by searching against public databases, using algorithms such as FASTA, BLAST, etc.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or expression construct” refer to nucleotide sequences that are capable of affecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced, but have been obtained from another cell or synthetically or recombinantly produced. Generally, though not necessarily, such nucleic acids encode proteins, i.e. exogenous proteins, that are not normally produced by the cell in which the DNA is transcribed or expressed. Similarly exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

The “specific activity” of an enzyme is herein understood to mean the amount of activity of a particular enzyme per amount of total host cell protein, usually expressed in units of enzyme activity per mg total host cell protein. In the context of the present invention, the specific activity of a particular enzyme may be increased or decreased as compared to the specific activity of that enzyme in an (otherwise identical) wild type host cell.

“Aerobic conditions” “Oxic conditions” or an aerobic or oxic fermentation process is herein defined as conditions or a fermentation process run in the presence of oxygen and in which oxygen is consumed, preferably at a rate of at least 0.5, 1, 2, 5, 10, 20 or 50 mmol/L/h, and wherein organic molecules serve as electron donor and oxygen serves as electron acceptor.

“Anaerobic or anoxic conditions” or an “anaerobic or anoxic fermentation process” is herein defined as conditions or a fermentation process run substantially in the absence of oxygen and wherein organic molecules serve as both electron donor and electron acceptors. Under anoxic conditions substantially no oxygen is consumed, preferably less than 5, 2, 1, or 0.5 mmol/L/h, more preferably 0 mmol/L/h is consumed (i.e. oxygen consumption is not detectable), or substantially no dissolved oxygen can be detected in the fermentation medium, preferably the dissolved oxygen concentration in the medium is less than 2, 1, 0.5, 0.2, 0.1% of air saturation, i.e. below the detection limit of commercial oxygen probes.

Any reference to nucleotide or amino acid sequences accessible in public sequence databases herein refers to the version of the sequence entry as available on the filing date of this document.

### DETAILED DESCRIPTION OF THE INVENTION

Several approaches have been followed in metabolically engineering S. cerevisiae with the aim of reducing glycerol formation in order to increase the production of ethanol from a certain amount of sugar. Although some successes have been obtained, the current methodologies are far from optimal. Hence, other options are urgently required in significantly improving the overall ethanol production process. Furthermore, improvements that stem from genetic engineering have both practical and legislative limitations.

The present inventors have now found a surprisingly new and integrated approach that not only reduces glycerol production with a concomitant higher ethanol output per amount of sugar. At the same time the invention effectively reduces the frequency of infections in the fermentations and furthermore it results in reaching higher final ethanol concentrations. The underlying basic principle is using acetaldehyde at various places and at various moments in time during the process in a well-controlled manner. Advantageously the approach is not limited to using only genetically modified organisms (GMO) but can be applied to non-GMOs as well.

1. Externally Supplied Acetaldehyde in Ethanol Fermentations

In a first aspect, the invention pertains to a process for producing ethanol. The process preferably comprises the steps of: a) fermenting a medium with a yeast cell in a fermenter, whereby the medium contains or is fed with: i) a source of a fermentable carbohydrate and ii) a source of acetaldehyde, and whereby the yeast cell ferments the fermentable carbohydrate and the acetaldehyde to ethanol; and, b) optionally, recovery of the ethanol from the medium.

Preferably in the process, acetaldehyde is present in the medium to reduce the formation of the by-product glycerol. Yeasts like S. cerevisiae produce glycerol in the absence of oxygen as a means of closing their redox balance. Under anoxic growth conditions, dissimilation of glucose to ethanol via glycolysis and pyruvate decarboxylation yields 2 ATP for each molecule of glucose converted to two molecules of ethanol and is NAD(H) neutral. But in the production of yeast biomass, a net formation of NADH from NAD⁺ occurs. The yeast keeps it redox balance neutral by reoxidizing NADH via the energy-consuming reduction of sugar to glycerol.

In the process of the invention, acetaldehyde is externally supplied to the yeast, which the organism will be able to use for balancing its redox situation by reducing the aldehyde to ethanol while reoxidizing NADH, instead of using the production of glycerol for this purpose. Most of the glycerol is produced at higher growth rates, i.e. prior to the ethanol-stress induced slow-down of the growth rate at higher ethanol concentrations, e.g. in a later phase of the fermentation process. The acetaldehyde is therefore most effectively applied for reducing the formation of glycerol at a stage in the fermentation process before the ethanol in the medium reaches a concentration that slows down the yeast's growth rate. Preferably, therefore, in the process of the invention, the acetaldehyde is present in or fed into the medium at least prior to the ethanol in the medium has a concentration that is higher than 50, 49, 45, 44, 40, 35, 30, 20, 10, or 5 kg/m³.

Alternatively and/or additionally, in the process of the invention, preferably the acetaldehyde is present in or fed into the medium at least prior to when the fermentation (i.e. yeast cell) enters stationary phase. Stationary phase is herein understood as the phase in the fermentation process wherein there is substantially no growth of the yeast cell(s). More preferably, the acetaldehyde is present in or fed into the medium at least during a stage in the process when the growth rate of the yeast cell is at least 0.005, 0.01, 0.02, 0.05, 0.1 h⁻¹, and/or prior to when the growth rate of the yeast cell is or slows down to a rate of no more than 0.1, 0.05, 0.02, 0.01, 0.005 h⁻¹.

Acetaldehyde is toxic to microbes including yeasts like S. cerevisiae. At the same time, acetaldehyde is released from yeast cells at a low rate during fermentation processes, e.g. in the final fermented mash of ethanol fermentations, its concentrations range between almost zero and 0.04 kg/m³. Preferably, in the process of the invention, the concentration of acetaldehyde in the medium is monitored and controlled not to exceed a maximum concentration and preferably to be within a certain range. Preferably, the concentration of acetaldehyde in the medium is controlled to be no more than 2.0, 1.5, 1.0, 0.75, 0.50, 0.35, 0.25, 0.22, 0.20, 0.18, 0.17, 0.16, 0.15, 0.14, 0.13, 0.12, 0.11, 0.10, 0.08, 0.06, 0.05, 0.044, 0.033, 0.029, 0.026, 0.024, 0.022, 0.020, 0.018, 0.015, 0.014, 0.013, 0.012 kg/m³. The concentration of acetaldehyde in the medium is further preferably controlled to be at least 0.0009, 0.0010, 0.0011, 0.0012, 0.0013, 0.0014, 0.0015, 0.0016, 0.0018, 0.0020, 0.0022, 0.0025, 0.0027, 0.0028, 0.0030 0.0032, 0.0036, 0.0040, 0.0044, 0.005, 0.007, 0.008, 0.009, 0.010, 0.011, 0.012, 0.013, 0.014, 0.015, 0.016, 0.018, 0.020, 0.022, 0.025, 0.027, 0.028, 0.030 0.032, 0.036, 0.040, 0.044, 0.05, 0.07, 0.09, 0.12, 0.16, 0.20, 0.35, or 0.50 kg/m³. Preferably, the rate of acetaldehyde fed into the medium is controlled to maintain the concentration of acetaldehyde in the medium to be no more than 2.0, 1.5, 1.0, 0.75, 0.50, 0.35, 0.25, 0.22, 0.20, 0.18, 0.17, 0.16, 0.15, 0.14, 0.13, 0.12, 0.11, 0.10, 0.08, 0.06, 0.05, 0.044, 0.033, 0.029, 0.026, 0.024, 0.022, 0.020, 0.018, 0.015, 0.014, 0.013, 0.012 kg/m³, and/or at least 0.0009, 0.0010, 0.0011, 0.0012, 0.0013, 0.0014, 0.0015, 0.0016, 0.0018, 0.0020, 0.0022, 0.0025, 0.0027, 0.0028, 0.0030 0.0032, 0.0036, 0.0040, 0.0044, 0.005, 0.007, 0.008, 0.009, 0.010, 0.011, 0.012, 0.013, 0.014, 0.015, 0.016, 0.018, 0.020, 0.022, 0.025, 0.027, 0.028, 0.030 0.032, 0.036, 0.040, 0.044, 0.05, 0.07, 0.09, 0.12, 0.16, 0.20, 0.35, or 0.50 kg/m³.

Preferably in the process of the invention, the amount of acetaldehyde that is fed into the medium (for consumption by the yeast) is at least one of: a) 1, 2, 4, 6, 8, 10, 15 or 20% on a molar basis, of the amount of carbohydrate (hexose and/or pentose) that is contained or fed into the medium, and preferably consumed by the yeast; and b) 0.5, 1, 2, 3, 4, 5, 8 or 10% on a molar basis, of the amount of ethanol that is produced by the yeast. Preferably, these relative amounts of acetaldehyde fed in relation to the carbohydrate consumed or ethanol produced apply to the overall process or they apply to the phase of the process prior to the ethanol in the medium having a concentration that is higher than 50, 49, 45, 44, 40, 35, 30, 20, 10, or 5 kg/m³.

In one embodiment, the process of the invention has two phases, wherein in a first phase of the process, the ethanol concentration in the medium is below a threshold concentration, and in a second phase, the ethanol concentration in the medium is above that threshold concentration. The threshold ethanol concentration in the medium preferably is higher than 80, 60, 50, 40, 30, 20, 10, or 5 kg/m³. More preferably the threshold ethanol concentration is a range of ethanol concentrations with a lower limit of 80, 60, 50, 40, 30, 20, 10, or 5 kg/m³ and an upper limit of 100, 90, 80, 70, 60, 50 or 40 kg/m³. In this embodiment, the first phase of the process is a phase wherein acetaldehyde is externally supplied to the medium to reduce the formation of glycerol as by-product, as described hereinabove. Preferably, therefore, in the first phase of the process, the acetaldehyde concentration in the medium is controlled to be no more than 2.0, 1.5, 1.0, 0.75, 0.50, 0.35, 0.25, 0.22, 0.20, 0.18, 0.17, 0.16, 0.15, 0.14, 0.13, 0.12, 0.11, 0.10, 0.08, 0.06, 0.05, 0.044, 0.033, 0.029, 0.026, 0.024, 0.022, 0.020, 0.018, 0.015, 0.014, 0.013, 0.012 kg/m³, and/or at least 0.0009, 0.0010, 0.0011, 0.0012, 0.0013, 0.0014, 0.0015, 0.0016, 0.0018, 0.0020, 0.0022, 0.0025, 0.0027, 0.0028, 0.0030 0.0032, 0.0036, 0.0040, 0.0044, 0.005, 0.007, 0.008, 0.009, 0.010, 0.011, 0.012, 0.013, 0.014, 0.015, 0.016, 0.018, 0.020, 0.022, 0.025, 0.027, 0.028, 0.030 0.032, 0.036, 0.040, 0.044, 0.05, 0.07, 0.09, 0.12, 0.16, 0.20, 0.35, or 0.50 kg/m³.

In this embodiment, in the second phase of the process, higher ethanol concentrations are present in the medium, which affect the performance of the yeast cell in terms of both growth and ethanol production. Barber et al. (2002, supra) and Vriesekoop et al. (2007, supra) have reported that acetaldehyde when added at low concentration has a beneficial effect on the performance of yeast at high ethanol levels. Preferably, therefore, in the second phase of the process, the acetaldehyde concentration in the medium is controlled to be no more than 0.3 kg/m³. Preferably, in the second phase of the process the acetaldehyde concentration in the medium is controlled to be at least 0.01, 0.02, or 0.05 kg/m³ and no more than 0.1, 0.2, or 0.3 kg/m³.

Preferably in the processes of the invention, in a first phase of the process, when the growth rate of the yeast is higher, acetaldehyde is fed into the medium at a specific acetaldehyde consumption rate of at least 0.005, 0.01, 0.02, 0.04, 0.08 or 0.15 g acetaldehyde/g cells (dry weight)/hour. Preferably these rates are the average acetaldehyde consumption rates over the first phase. Next, in a second phase, when the growth rate of the yeast is lower, no acetaldehyde is fed, or acetaldehyde is fed into the medium at a specific acetaldehyde consumption rate of at least 0.0005, 0.001, 0.002, 0.004, 0.008 or 0.015 g acetaldehyde/g cells (dry weight)/hour. Preferably these rates are the average acetaldehyde consumption rates over the second phase. Preferably, the first phase of the process is herein understood as the phase wherein the ethanol concentration in the medium is below a threshold concentration, and in the second phase, the ethanol concentration in the medium is above that threshold concentration, whereby the threshold concentrations preferably are as defined above.

In a further embodiment, the fact that yeasts like S. cerevisiae are less sensitive to acetaldehyde compared to many other microorganisms such as e.g. lactic acid bacteria, is used to suppress growth of infections during the fermentation process. In this embodiment, the acetaldehyde concentration in the medium is preferably controlled as indicated above in relation to the ethanol concentration because at lower ethanol concentrations higher concentration of acetaldehyde will be required for suppression of infections. Higher ethanol concentrations themselves will already suppress of infections and less acetaldehyde will be required for suppression of infections.

In the process of the invention, the source of acetaldehyde supplied to the medium can be a source in liquid form or in gaseous form, or a combination thereof. Acetaldehyde (systematic name ethanal) is a colorless liquid with a molar mass of 44 g mol⁻¹. It has a liquid density of 0.78 g·cm³ and a boiling point of only 20° C. Acetaldehyde is soluble in water in all proportions. The values for Henry's law constant k_{H} for acetaldehyde as taken herein is 15 M/atm, although variations in reported values occur in the literature. The definition for this constant is k_{H}=Cₐ/P_{g} in which Cₐ is the acetaldehyde concentration in water and P_{g} is the partial pressure of acetaldehyde in the gas phase. As for the sake of comparison, k_{H} for oxygen is 1.3*10⁻³ M/atm, whereas k_{H} for ethanol is approximately 200 M/atm. The density of acetaldehyde in gas at 1 atmosphere just above its boiling point is taken as 1.8 kg/m³.

When supplied in liquid form, the acetaldehyde is preferably diluted in an aqueous solution so as to minimize toxic effects of local high concentrations in the medium at the point(s) where the acetaldehyde is supplied into in the fermenter/medium.

Alternatively the acetaldehyde is supplied in gaseous form, in which case the acetaldehyde is preferably mixed with at least a part of an (carbon dioxide-containing) off-gas stream from the fermenter. In this embodiment, part of the off-gas stream is recycled back into the fermenter after being mixed with gaseous acetaldehyde.

Appropriate means for supplying the acetaldehyde to the medium and controlling the rate of the acetaldehyde supplied to the medium, either in liquid or gaseous form, are well known in the art per se (see also below).

The concentrations of acetaldehyde and/or ethanol in the medium can be monitored by methods well known in the art per se. Preferably, in the processes of the invention the concentration in the medium of at least one of acetaldehyde and ethanol is monitored on-line in a gas (carbon dioxide) exhaust or off-gas streams from the fermenter, e.g. using a mass spectrometer or a gas chromatograph with flame ionization detector (FID). On-line methodologies are available for selective measurement of acetaldehyde and/or ethanol in e.g. CO₂ exhaust streams from the fermenter, at detection limits of <30 part-per-billion (see e.g. GOW-MAC Instrument Co. at www.gow-mac.com), allowing for good process monitoring and control for administration of acetaldehyde to the fermentation system.

Standard methods and means of process control in dosing acetaldehyde supply into the medium can be applied. For example, optionally measurements derived from off-gas measurements, are compared to setpoints to adjust the dosing of acetaldehyde to achieve setpoint concentrations.

In a process of the invention, a source of a fermentable carbohydrate (and acetaldehyde) are fermented to ethanol by a yeast cell. The source of fermentable carbohydrate comprises or consists of carbohydrates that are fermentable by a yeast cell of the invention, which yeast cell can be a yeast cell modified to have the ability to ferment pentoses such as xylose and arabinose. A suitable source of fermentable carbohydrate therefore comprises or consists of at least one of hexose and pentoses, including but not limited to glucose, fructose, sucrose, maltose, raffinose, galactose, xylose, arabinose and mannose. Various substrates or various type of plant materials can be used source of fermentable carbohydrate in the processes of the invention for producing ethanol. For example, common used feedstocks include corn (dry meal or wet meal), wheat, sugar beet, sugar cane and molasses. Other types of plant material can also be used as the feedstock, including e.g. lignocellulosic fractions of plant biomass for production of second generation bioethanol.

In the process of the invention, the sources of hexoses and pentoses may be hexoses and/or pentoses as such (i.e. as monomeric sugars) or they may be in the form of any carbohydrate oligo- or polymer comprising hexoses and/or pentoses units, such as e.g. lignocellulose, arabinans, xylans, cellulose, starch, inulin, and the like. For release of hexoses and/or pentoses units from such carbohydrates, appropriate carbohydrases (such as arabinases, xylanases, glucanases, (gluco)amylases, cellulases, glucanases, inulinases and the like) may be added to the fermentation medium or may be produced by a yeast cell to this end. In the latter case the modified yeast cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases preferably during the fermentation. This, in turn, will prevent repression of systems required for metabolism and transport of non-glucose sugars such as xylose and arabinose. In a preferred process the modified host cell ferments both the glucose and the pentoses, preferably simultaneously in which case preferably a modified yeast cell is used which is insensitive to glucose repression to prevent diauxic growth. In addition to a source of fermentable carbohydrate as carbon and energy source, the fermentation medium will further comprise the appropriate ingredients required for growth of the yeast cell of the invention. Compositions of fermentation media for growth of yeasts are well known in the art.

The fermentation process is preferably run at a temperature that is optimal for the yeast cells of the invention. Thus, for most yeasts cells, the fermentation process is performed at a temperature which is less than 42° C., preferably less than 38° C. For yeast cells, the fermentation process is preferably performed at a temperature which is lower than 35, 33, 30 or 28° C. and at a temperature which is higher than 20, 22, or 25° C.

The fermentation process is preferably run at a neutral or acidic pH, preferably at a pH in the range of 2-7, more preferably at a pH in the range of 2.5-6, and most preferably a pH in the range of 3.0-5.5 (as measured in the fermentation medium at the temperature at which fermentation takes place).

The fermentation process preferably is an anoxic or anaerobic fermentation process as defined hereinabove. Anoxic processes of the invention are preferred over aerobic processes because anaerobic processes do not require investments and energy for aeration and in addition, anaerobic processes produce higher product yields than aerobic processes. An anoxic fermentation process of the invention does not exclude that some air/oxygen is blown into the medium that the yeast cell can use e.g. for oxidase-dependent biosynthetic reactions. However, under these circumstances, substantially no dissolved oxygen can be detected in the fermentation medium, i.e. the dissolved oxygen concentration in the medium is less than 2, 1, 0.5, 0.2, 0.1% of air saturation, and preferably below the detection limit of commercial oxygen probes in anoxic ethanol fermentation.

Preferably in the processes of the invention, the volumetric ethanol productivity is preferably at least 0.5, 1.0, 1.5, 2.0, 2.5, 3.0, 5.0 or 10.0 kg ethanol per m³ per hour. The ethanol yield on fermentable carbohydrate (hexose or pentose) and/or acetaldehyde in the process preferably is at least 50, 60, 70, 80, 90, 95 or 98%. The ethanol yield is herein defined as a percentage of the theoretical maximum yield, which, for hexose and pentose is 0.51 g. ethanol per g. hexose or pentose. For acetaldehyde the theoretical maximum yield is 1.05 g. ethanol per g. acetaldehyde.

Preferably in the process of the invention, the amount of glycerol produced is less than 5, 2, 1, 0.5, or 0.3 or 0.1% of the carbon consumed on a molar basis. Preferably in the process of the invention, the amount of acetic acid produced is less than 5, 2, 1, 0.5, or 0.3 or 0.1% of the carbon consumed on a molar basis. Preferably in the process of the invention, the amount of pyruvic acid produced is less than 5, 2, 1, 0.5, or 0.3 or 0.1% of the carbon consumed on a molar basis. Preferably in the process of the invention, the amount of butanediol-2,3 produced is less than 5, 2, 1, 0.5, or 0.3 or 0.1% of the carbon consumed on a molar basis.

Preferably in the process of the invention, the amount of glycerol produced is reduced by at least 15, 20, 25, 30, 40, 50, 60, 70 or 80%, as compared to an identical process wherein no external acetaldehyde is supplied during the fermentation process.

The process of the invention preferably is a batch process, a fed-batch process or a multistage continuous fermentation process (see e.g. Ingledew, in “The Alcohol Textbook”, 4^{th} edition, editors K. A. Jacques, T. P. Lyons and D. R Kensall, pages 135-143, 2003, Nottingham University Press, UK). The process of the invention preferably is not a single stage continuous process, such as e.g. a chemostat.

The process of the invention further preferable comprises a step b) for recovery of the ethanol from the fermented medium (also referred to as “beer”), obtained in step a) of the process. Recovery of ethanol from the fermented medium is performed by methods well known in the art such e.g. by distillation. Means and methods for recovery of ethanol by distillation from fermented media are e.g. described by Madson (“The Alcohol Textbook”, 4^{th} edition, editors K. A. Jacques, T. P. Lyons and D. R Kensall, pages 319-336, 2003, Nottingham University Press, UK). The ethanol may further be dehydrated using a molecular sieve e.g. as described by Bibb Swain (“The Alcohol Textbook”, 4^{th} edition, editors K. A. Jacques, T. P. Lyons and D. R Kensall, pages 337-341, 2003, Nottingham University Press, UK). The recovery of ethanol by distillation produces a stream of ethanol.

2. The Yeast Cell

In a second aspect, the invention pertains to a yeast cell for fermenting a fermentable carbohydrate and optionally acetaldehyde to ethanol in a process according to the invention.

Yeasts are herein defined as eukaryotic microorganisms and include all species of the subdivision Eumycotina (Yeasts: characteristics and identification, J. A. Barnett, R. W. Payne, D. Yarrow, 2000, 3rd ed., Cambridge University Press, Cambridge UK; and, The yeasts, a taxonomic study, C P. Kurtzman and J. W. Fell (eds) 1998, 4th ed., Elsevier Science Publ. B. V., Amsterdam, The Netherlands) that predominantly grow in unicellular form. Yeasts may either grow by budding of a unicellular thallus or may grow by fission of the organism.

Preferably the yeast cell of the invention is a yeast cell that is naturally capable of anoxic fermentation, more preferably alcoholic fermentation and most preferably anoxic alcoholic fermentation.

A preferred yeast cell of the invention belongs to one of the genera Saccharomyces, Kazachstania and Naumovia (Kurtzman, 2003, FEMS Yeast Research 4:233-245). More preferably, a yeast cell of the invention belongs to a species selected from the group consisting of Saccharomyces cerevisiae S. bayanus, S. bulderi, S. cervazzii, S. cariocanus, S. castellii, S. dairenensis, S. exiguus, S. kluyveri, S. kudriazevii, S. mikatae, S. paradoxus, S. pastorianus, S. turicensis, S. unisporus (Kurtzman, 2003, supra; and J. A. Barnett, R. W. Payne, D. Yarrow, 2000, supra).

Particularly when compared to bacteria, yeasts, from these genera, have many attractive features for industrial fermentative processes for producing ethanol, including e.g. their high tolerance to acids, ethanol and other harmful compounds, their high osmo-tolerance and their capability of anoxic growth, and of course their high fermentative capacity.

Suitable strains of yeast cell for use in the invention include e.g. strains described in van Dijken et al. (2000, Enzyme and Microbial Technology 26:706-714) such as e.g. S. cerevisiae CEN.PK2-1C and related CEN.PK strains and S. cerevisiae CBS 8066. More suitable are industrial yeast strains such as the commercial strains Gert Strand Turbo yeasts, Alltech SuperStart™, Fermiol Super HA™, Thermosacc™ and Ethanol Red™. Also suitable are yeast cells derived from any of these strain by modifications as described herein below.

A preferred yeast cell for use in the processes of the invention contains an active glycolysis. The yeast cell further preferably has a high tolerance to ethanol, a high tolerance to low pH (i.e. capable of growth at a pH lower than 5, 4, or 3) and towards organic acids like lactic acid, acetic acid, propionic acid, butyric acid and/or formic acid and sugar degradation products such as furfural and hydroxymethylfurfural, and a high tolerance to elevated temperatures. Any of these characteristics or activities of the yeast cell may be naturally present in the yeast cell or may be introduced or modified by genetic modification, preferably by self cloning or by the methods of the invention described below.

A suitable yeast cell is a cultured cell, a yeast cell that may be cultured in fermentation process, preferably in a submerged fermentation process.

In one aspect of the invention pertains to a yeast cell for use in the processes of the invention. In one embodiment the yeast cell is a non-GMO, i.e. a yeast strain that is not genetically modified organism. A non-GMO yeast cell is understood to be a cell that contains no genetic modifications that are the result of genetic engineering, using e.g. recombinant DNA technology and/or synthetic DNA. In particular a non-GMO yeast cell contains no heterologous DNA sequences. However, the non-GMO yeast cell can contain genetic modifications that are the result of spontaneous and/or induced random mutations. In particular “evolutionary engineering” can be applied to obtain non-GMO yeast cell in accordance with the invention ( [symbol] akar et al. 2011. FEMS Yeast Research 12:171-182).

A preferred yeast cell of the invention is a yeast cell modified to have increased resistance to acetaldehyde as compared to a corresponding unmodified parent strain. Preferably, the yeast cell modified with increased resistance to acetaldehyde is obtained by evolutionary engineering. This approach is successfully followed by the inventors in arriving at strains that are more resistant to acetaldehyde than the parent strain.

In another embodiment the yeast cell is a genetically modified yeast cell.

2.2 Methods for Modifying and Constructing the Yeast Cells of the Invention

For the genetic modification of the yeast cells of the invention, standard genetic and molecular biology techniques are used that are generally known in the art and have e.g. been described by Sambrook and Russell (2001, “Molecular cloning: a laboratory manual” (3rd edition), Cold Spring Harbor Laboratory, Cold Spring Harbor Laboratory Press) and Ausubel et al. (1987, eds., “Current protocols in molecular biology”, Green Publishing and Wiley Interscience, New York). Furthermore, the construction of genetically modified (yeast) host strains may be carried out by genetic crosses, sporulation of the resulting diploids, tetrad dissection of the haploid spores containing the desired auxotrophic markers, and colony purification of such haploid host cells in the appropriate selection medium. All of these methods are standard yeast genetic methods known to those in the art. See, for example, Sherman et al, Methods Yeast Genetics, Cold Spring Harbor Laboratory, NY (1978) and Guthrie et al. (Eds.) Guide To Yeast Genetics and Molecular Biology Vol. 194, Academic Press, San Diego (1991).

In general, suitable promoters for the expression of the heterologous nucleotide sequence coding for desired enzyme activities and/or for overexpression of endogenous genes in the context of the invention, include promoters that are preferably insensitive to catabolite (glucose) repression, that are active under oxic and under anoxic conditions and/or that preferably do not require specific carbon sources for induction. Promoters having these characteristics are widely available and known to the skilled person. Suitable examples of such promoters include e.g. promoters from glycolytic genes such as the phosphofructokinase (PFK), triose phosphate isomerase (TPI), glyceraldehyde-3-phosphate dehydrogenase (GPD, TDH3 or GAPDH), pyruvate kinase (PYK), phosphoglycerate kinase (PGK), glucose-6-phosphate isomerase promoter (PGII) promoters from yeasts. More details about such promoters from yeast may be found in (WO 93/03159). Other useful promoters are ribosomal protein encoding gene promoters (TEFI), alcohol dehydrogenase promoters (ADH1, preferably a modified (constitutive) version of the ADH1 promoter (SEQ ID NO: 1), ADH4, and the like), the enolase promoter (ENO) and the hexose(glucose) transporter promoter (HXT7). Alternatively, a suitable promoter for these purposes is a promoter that allows (over)expression under anoxic conditions. A preferred example of such an anoxic promoter is e.g. the S. cerevisiae ANB1 promoter (SEQ ID NO: 2). Other promoters, both constitutive and inducible, and enhancers or upstream activating sequences will be known to those of skill in the art. Preferably the promoter that is operably linked to nucleotide sequence as defined above is homologous to the yeast cell. Suitable terminator sequences are e.g. obtainable from the cytochrome cl (CYC1) gene or an alcohol dehydrogenase gene (e.g. ADH1).

To increase the likelihood that the enzymes are expressed at sufficient levels and in active form in the transformed yeast cells of the invention, the nucleotide sequence encoding of enzymes of the invention, are preferably adapted to optimize their codon usage to that of the yeast cell in question. The adaptiveness of a nucleotide sequence encoding an enzyme to the codon usage of a yeast cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al, 2003, Nucleic Acids Res. 3J_(8):2242-51). An adapted nucleotide sequence preferably has a CAI of at least 0.2, 0.3, 0.4, 0.5, 0.6, 0.7, 0.8 or 0.9. Most preferred are the sequences which have been codon optimized for expression in the yeast cell in question such as e.g. S. cerevisiae cells.

There are various means available in the art for increasing a specific enzymatic activity in a cell of the invention. In particular, the specific activity of an enzyme can be increased by overexpressing a gene coding for the enzyme, e.g. by increasing the copy number of a gene coding for the enzyme in the cell, as can be achieved e.g. by increasing the copy number of the gene (i.e. increasing the gene dosage) in the cell by integrating additional copies of the gene in the cell's genome, by expressing the gene from an episomal multicopy expression vector or by introducing a episomal expression vector that comprises multiple copies of the gene. Preferably, in the context of the present invention, gene dosage is increased by integrating additional copies of the gene in the cell's genome. Alternatively overexpression of enzymes in the host cells of the invention can be achieved by using a heterologous stronger promoter than the promoter that is native to the sequence coding for the enzyme to be overexpressed. Although the promoter preferably is heterologous to the coding sequence to which it is operably linked, it is also preferred that the promoter is homologous, i.e. endogenous to the cell. Preferably the heterologous promoter is capable of producing a higher steady state level of the transcript comprising the coding sequence (or is capable of producing more transcript molecules, i.e. mRNA molecules, per unit of time) than is the promoter that is native to the coding sequence, preferably under anoxic conditions when grown on glucose as (sole) carbon sources.

Many methods for modifying endogenous target genes in yeast cells so as to reduce or eliminate the activity of the encoded target proteins are known to one skilled in the art and may be used for modifying the yeast cells of the invention. Modifications that may be used to reduce or eliminate expression of a target protein are disruptions that include, but are not limited to, deletion of the entire gene or a portion of the gene encoding the target protein, inserting a DNA fragment into the target gene (in either the promoter or coding region) so that the protein is not expressed or expressed at lower levels, introducing a mutation into the target coding region which adds a stop codon or frame shift such that a functional protein is not expressed, and introducing one or more mutations into a target coding region to alter amino acids so that a non-functional target protein, or a target protein with reduced enzymatic activity is expressed. In addition, expression of the target gene may be blocked by expression of an antisense RNA or an interfering RNA, and constructs may be introduced that result in co-suppression. Moreover, a target coding sequence may be synthesized whose expression will be low because rare codons are substituted for plentiful ones, when this suboptimal coding sequence is substituted for the corresponding endogenous target coding sequence. Preferably such a suboptimal coding sequence will have a codon adaptation index (see above) of less than 0.5, 0.4, 0.3 0.2, or 0.1. Such a suboptimal coding sequence will produce the same polypeptide but at a lower rate due to inefficient translation. In addition, the synthesis or stability of the transcript may be reduced by mutation. Similarly the efficiency by which a protein is translated from mRNA may be modulated by mutation, e.g. by using suboptimal translation initiation codons. All of these methods may be readily practiced by one skilled in the art making use of the known or identified sequences encoding target proteins.

DNA sequences flanking a target coding sequence are also useful in some modification procedures and are available for yeasts such as for Saccharomyces cerevisiae in the complete genome sequence coordinated by Genome Project ID9518 of Genome Projects coordinated by NCBI (National Center for Biotechnology Information) with identification GOPID #13838. In particular, DNA sequences surrounding a target coding sequence are useful for modification methods using homologous recombination. For example, in this method sequences flanking the target gene are placed on either site of a selectable marker gene to mediate homologous recombination whereby the marker gene replaces the target gene. Also partial target gene sequences and target gene flanking sequences bounding a selectable marker gene may be used to mediate homologous recombination whereby the marker gene replaces a portion of the target gene. In addition, the selectable marker may be flanked by site-specific recombination sites, so that following expression of the corresponding site-specific recombinase, the resistance gene is excised from the genomic locus where the target gene was present without reactivating the latter. The site-specific recombination leaves behind a recombination site which disrupts expression of the target protein. The homologous recombination vector may be constructed to also leave a deletion in the target gene following excision of the selectable marker, as is well known to one skilled in the art.

Deletions may be made using mitotic recombination as described in Wach et al. ((1994) Yeast 10:1793-1808). This method involves preparing a DNA fragment that contains a selectable marker between genomic regions that may be as short as 20 bp, and which bound, i.e. flank the target DNA sequence. This DNA fragment can be prepared by PCR amplification of the selectable marker gene using as primers oligonucleotides that hybridize to the ends of the marker gene and that include the genomic regions that can recombine with the yeast genome. The linear DNA fragment can be efficiently transformed into yeast and recombined into the genome resulting in gene replacement including with deletion of the target DNA sequence (as described in Methods in Enzymology, 1991, 194:281-301). Moreover, promoter replacement methods may be used to exchange the endogenous transcriptional control elements allowing another means to modulate expression such as described in Mnaimneh et al. ((2004) Cell 118(1):31-44).

2.3 Reducing Acetaldehyde Dehydrogenase Activity

In one embodiment of the invention, the yeast cell used in the invention is modified so as to avoid or reduce the synthesis of acetate from acetaldehyde. Preferably, therefore, the yeast cell comprises a genetic modification that reduces or eliminates endogenous specific acetaldehyde dehydrogenase activity in the cell, more preferably the specific cytosolic acetaldehyde dehydrogenase activity is reduced or eliminated in the cell.

In the yeast genome, there are five genes known to encode aldehyde dehydrogenases, as well as an additional gene with sequence similarity. Ald2p and Ald3p are cytosolic enzymes which use only NAD⁺ as cofactor (EC 1.2.1.5). Both genes are induced in response to ethanol or stress and repressed by glucose. Ald4p and Ald5p are mitochondrial, use NAD and NADP as cofactors, and are K⁺ dependent. Ald4p, the major isoform, is glucose repressed and ald4 mutants do not grow on ethanol, while Ald5p, the minor isoform, is constitutively expressed. ALD6 encodes the Mg²⁺ activated cytosolic enzyme, which uses NADP⁺ as cofactor and is constitutively expressed (EC 1.2.1.4). The cytosolic ALD6 gene product is the major enzyme responsible for catalyzing the oxidation of acetaldehyde to acetate in yeast.

Thus, in a yeast cell of the invention, the gene to be modified for reducing or eliminating the specific acetaldehyde dehydrogenase activity in the cell is one or more or all of the ALD1, ALD2, ALD3, ALD4, ALD5 and ALD6 genes or their corresponding orthologues. More preferably, the cell is modified such that at least the expression of the S. cerevisiae ALD6 gene, encoding the amino acid sequence of SEQ ID NO: 3, or an orthologue thereof in another species is reduced or eliminated. Therefore, a gene to be modified for reducing or eliminating the specific acetaldehyde dehydrogenase activity in the cell of the invention, preferably is a gene encoding a amino acid sequence with at least 70, 75, 80, 85, 90, 95, 98 or 99% sequence identity to SEQ ID NO: 3.

In the yeast cells of the invention, the specific acetaldehyde dehydrogenase activity is preferably reduced by at least a factor 0.8, 0.5, 0.3, 0.1, 0.05 or 0.01 as compared to cells of a strain which is genetically identical except for the genetic modification causing the reduction in activity, at least under anoxic conditions.

Methods for reducing or eliminating the specific cytosolic acetaldehyde dehydrogenase activity in the cell of the invention are as described above in 2.2. S. cerevisiae strains with (a) deletion(s) of the ALD6 gene(s) can be constructed as previously described by Saint-Prix, et al. (2004. Microbiology 150:2209-2220).

2.4 Reducing NADH-Dependent Glycerol Synthesis

Preferably, the yeast cell of the invention has a genetic modification whereby NADH-dependent glycerol synthesis is reduced. Preferably, NADH-dependent glycerol synthesis is not completely eliminated in the yeast cell. The NADH-dependent glycerol synthesis is preferably reduced by at least a factor 0.8, 0.5, 0.3, 0.1, 0.05 or 0.01 as compared to cells of a strain which is genetically identical except for the genetic modification causing the reduced glycerol synthesis, at least under anoxic conditions. NADH-dependent glycerol synthesis can be reduced by reducing or eliminating at least one of the specific glycerol-3-phosphate dehydrogenase and glycerol-3-phosphatase activities in the yeast cell of the invention.

Thus, in one embodiment, the specific glycerol-3-phosphate dehydrogenase (EC 1.1.1.8) activity is reduced or eliminated in the yeast cell to prevent or reduce the formation of glycerol as by-product. Yeast strains may have one or more (different) genes encoding NAD-dependent glycerol-3-phosphate dehydrogenases. In S. cerevisiae, the GPD1 and GPD2 genes encode functional homologues of NAD-dependent glycerol-3-phosphate dehydrogenases.

In a preferred yeast cell of the invention, at least one the S. cerevisiae GPD1 and GPD2 genes, or at least one of their orthologues in another species, is genetically modified to reduce or eliminate the specific glycerol-3-phosphate dehydrogenase activity in the cell. Preferably, the gene that is modified for reducing or eliminating the glycerol-3-phosphate dehydrogenase activity in the cell, is a gene encoding a amino acid sequence with at least 70, 75, 80, 85, 90, 95, 98 or 99% sequence identity to at least one of SEQ ID NO's: 4 and 5 (the amino acid sequences of the S. cerevisiae GPD1 and GPD2 encoded glycerol-3-phosphate dehydrogenases, respectively).

In the yeast cells of the invention, the specific glycerol-3-phosphate dehydrogenase activity is preferably reduced by at least a factor 0.8, 0.5, 0.3, 0.1, 0.05 or 0.01 as compared to cells of a strain which is genetically identical except for the genetic modification causing the reduction in expression, at least under anoxic conditions.

In another embodiment, the specific glycerol-3-phosphatase activity (EC 3.1.3.21) is reduced or eliminated in the yeast cell to prevent or reduce the formation of glycerol as by-product. In S. cerevisiae glycerol-3-phosphatase activity is encoded by the endogenous HOR2 and RHR2 genes.

In a preferred yeast cell of the invention, at least one the S. cerevisiae HOR2 and RHR2 genes, or at least one of their orthologues in another species, is genetically modified to reduce or eliminate the specific glycerol-3-phosphatase activity in the cell. Preferably, the gene that is modified for reducing or eliminating the glycerol-3-phosphatase activity in the cell, is a gene encoding a amino acid sequence with at least 70, 75, 80, 85, 90, 95, 98 or 99% sequence identity to at least one of SEQ ID NO's: 6 and 7 (the amino acid sequences of the S. cerevisiae HOR2 (GPP2) and RHR2 (GPP1) encoded glycerol-3-phosphatases, respectively).

In the cells of the invention, the specific glycerol-3-phosphatase activity is preferably reduced by at least a factor 0.8, 0.5, 0.3, 0.1, 0.05 or 0.01 as compared to cells of a strain which is genetically identical except for the genetic modification causing the reduction in expression, at least under anoxic conditions.

Methods for reducing or eliminating at least one of the specific glycerol-3-phosphate dehydrogenase and glycerol-3-phosphatase activities in the yeast cell of the invention are as described above in 2.2. S. cerevisiae strains with deletions of one or both of the GPD1 and GPD2 genes can be constructed as previously described in WO2013/081456. S. cerevisiae strains with deletions of one or both of the GPP1/RHR2 and GPP2/HOR2 genes can be constructed as previously described by Pahlman et al. (2001. J Biol Chem. 276(5):3555-63) or Wojda et al. (2007. Arch Microbiol. 188(2):175-84).

2.5 Reducing Glycerol Transmembrane Transporter Activity

In another embodiment, the formation of glycerol as by-product is prevented or reduced by genetically modifying a plasma membrane channel involved in the efflux of glycerol from the yeast cell so as to reduce or eliminate its activity. Reduction of glycerol efflux from the cell leads to a decreased production of glycerol by feed-back regulation as glycerol accumulates within the cells, thereby reducing the carbon flux towards glycerol biosynthesis.

In a preferred yeast cell of the invention, the S. cerevisiae FSP1 gene (encoding a aquaglyceroporin, a plasma membrane glycerol channel involved in efflux of glycerol), or its orthologue in another species, is genetically modified to reduce or eliminate the efflux of glycerol from the cell. Preferably, the gene that is modified for reducing or eliminating the activity of the plasma membrane channel involved in the efflux of glycerol from the cell, is a gene encoding a amino acid sequence with at least 70, 75, 80, 85, 90, 95, 98 or 99% sequence identity to SEQ ID NO: 8 (the amino acid sequences of the S. cerevisiae FSP1 encoded aquaglyceroporin).

In the cells of the invention, the efflux of glycerol from the cell is preferably reduced by at least a factor 0.8, 0.5, 0.3, 0.1, 0.05 or 0.01 as compared to cells of a strain which is genetically identical except for the genetic modification causing the reduction in expression, preferably under anoxic conditions.

Methods for reducing or eliminating the activity of the plasma membrane channel involved in the efflux of glycerol from the cell of the invention are as described above in 2.2. S. cerevisiae strains with (a) deletion(s) of the FSP1 gene(s) were constructed as previously described by Wei et al. (2013. Appl Environ Microbiol. 79(10): 3193-3201).

2.6 Yeast Cells for Use in Second Generation Processes

In one embodiment, the yeast cell of the invention, is a yeast cell that is modified for use in second generation process for producing bioethanol from lignocellulosic feedstocks. Accordingly, the yeast cell is modified to have the ability to use pentoses as carbon and energy source. Preferably the yeast cell is modified to have the ability to anoxically grow on pentoses such as xylose and arabinose. As most wild type yeasts do not have the ability to anoxically ferment pentoses such as xylose and arabinose, a preferred yeast cell of the invention is a cell that has been modified to have this ability. Such modifications will at least include the expression of an exogenous xylose isomerase activity (for xylose) and/or expression of exogenous arabinose isomerase (araA), ribulokinase (araB), and ribulose-5-P-4-epimerase (araD) activities (for arabinose).

A preferred yeast cell of the invention therefore comprises a genetic modification that introduces into the cell at least one of: i) expression of an exogenous gene encoding a xylose isomerase (EC 5.3.1.5), which gene confers to the cell the ability to isomerize xylose into xylulose; and, ii) expression of exogenous genes coding for a L-arabinose isomerase (EC 5.3.1.4), a L-ribulokinase (EC 2.7.1.16) and a L-ribulose-5-phosphate 4-epimerase (EC 5.1.3.4), which genes together confer to the cell the ability to convert L-arabinose into D-xylulose 5-phosphate.

Yeast strains modified for the ability to directly isomerize xylose into xylulose have been described in e.g. WO 2003/062430, US 20060234364, Madhavan et al., 2008, DOI 10.1007/s00253-008-1794-6, WO 2006/009434, WO 2009/109633, Brat et al., 2009, Appl. Environ. Microbiol. 75: 2304-2311, WO 2010/070549, WO 2010/074577 and WO 2011/006136. Yeast strains modified for the ability to convert L-arabinose into D-xylulose 5-phosphate have been described in Wisselink et al. (2007, AEM Accepts, published online ahead of print on 1 Jun. 2007; Appl. Environ. Microbiol. doi:10.1128/AEM.00177-07), WO 2008/041840 and WO 2009/011591.

Further preferred genetic modifications that may improve the yeast cell's ability to anoxically ferment pentoses such as xylose and arabinose include: iii) an increase of xylulokinase activity (by overexpression of endogenous genes and/or introduction of heterologous genes; see e.g. WO 2003/062430); iv) a genetic modification that increases the flux of the (non-oxidative part of the) pentose phosphate pathway as described in WO 06/009434, e.g. by overexpression of one or more of the ribulose-5-phosphate isomerase, ribulose-5-phosphate 3-epimerase, transketolase and transaldolase genes; and, vi) reduction or inactivation of expression of unspecific aldose reductase activity, as described in WO 06/009434. In a preferred embodiment these genetic modifications are: iii) overexpression of the S. cerevisiae XKS1 gene or an orthologue thereof; iv) overexpression of one or more of the S. cerevisiae RPI1, RPE1, TKL1 and TAL1 genes or orthologues thereof; and, v) reduction or elimination of the expression of the S. cerevisiae GRE3 gene or an orthologue thereof. The yeast cell is further preferably (modified to be) capable of active or passive transport of pentoses (xylose and/or arabinose) into the cell.

2.7 Increasing Acetaldehyde Resistance

A preferred yeast cell of the invention is a yeast cell modified to have increased resistance to acetaldehyde as compared to a corresponding unmodified parent strain. Preferably, the yeast cell modified with increased resistance to acetaldehyde is obtained by evolutionary engineering as described in the Examples herein.

Alternatively, or in addition, a yeast cell of the invention can be modified to have increased resistance to acetaldehyde as compared to a corresponding unmodified parent strain, by introducing one or more of the following genetic modifications into the yeast cell.

Grey et al. (1996, Curr. Genet. 29:437-440) describe that increased expression of the ADH1-encoded NADH-dependent alcohol dehydrogenase on a multi copy plasmid in yeast confers increased resistance to aldehydes such as formaldehyde. In one embodiment, therefore, the yeast cell of the invention has a genetic modification whereby the specific NADH-dependent alcohol dehydrogenase activity (EC 1.1.1.1) in the cell is increased in order to increase resistance to acetaldehyde. The NADH-dependent alcohol dehydrogenase activity is preferably increased by at least a factor 1.05, 1.1, 1.2, 1.5, 2.0, 5.0, 10, 20 or 50 as compared to cells of a strain which is genetically identical except for the genetic modification causing the increased alcohol dehydrogenase activity, at least when grown on glucose under anoxic conditions.

NADH-dependent alcohol dehydrogenase activity can be increased by increasing the expression of one or more genes encoding an NADH-dependent alcohol dehydrogenase, e.g. by overexpression of an endogenous gene and/or expression of an exogenous gene. Suitable genes for overexpression are the S. cerevisiae genes that encode alcohol dehydrogenases involved in ethanol metabolism, ADH1 to ADH5, or orthologues thereof in other species. More preferred are genes encoding one or more of the four enzymes, Adh1p, Adh3p, Adh4p, and Adh5p, which reduce acetaldehyde to ethanol during glucose fermentation. Most preferred is, however, the ADH1 gene or an orthologue thereof from another species, encoding the major cytosolic enzyme responsible for catalyzing the reduction of acetaldehyde to ethanol with the concomitant regeneration of NAD⁺ (EC 1.1.1.1), and overexpression of which is known to confer hyper-resistance to aldehydes in S. cerevisiae (Grey et al., 1996, supra).

Thus, a preferred gene to be modified for increasing the specific ADH1-encoded alcohol dehydrogenase activity in the cell of the invention is the S. cerevisiae ADH1 gene, encoding the amino acid sequence of SEQ ID NO: 9, or an orthologue thereof in another species. Therefore a gene to be overexpressed for increasing the specific ADH1-encoded alcohol dehydrogenase activity in the cell of the invention, preferably is a gene encoding an amino acid sequence with at least 70, 75, 80, 85, 90, 95, 98 or 99% sequence identity to SEQ ID NO: 9.

Alternatively or further, preferred genes to be modified for increasing the specific alcohol dehydrogenase activity in the cell of the invention are one or more of the S. cerevisiae ADH3, ADH4 and ADH5 genes or orthologues thereof in another species, preferably encoding an amino acid sequence with at least 70, 75, 80, 85, 90, 95, 98 or 99% sequence identity to the amino acid sequences of the S. cerevisiae ADH3, ADH4 and ADH5 genes (with respectively Genbank accession no.'s CAA89229.1, CAA64131.1 and CAA85103.1).

In another embodiment, for increasing the resistance to acetaldehyde the cell of the invention comprises a (further) genetic modification that increases the specific activity of the S. cerevisiae SFA1 gene, or an orthologue thereof in another species, encoding a bifunctional enzyme containing both NADH-dependent alcohol dehydrogenase and glutathione-dependent aldehyde dehydrogenase activities, as described in WO 2005/111214 and in Dickinson et al (2003, J. Biol Chem. 278(10):8028-34), disclosing a yeast strain with an increased ability of reducing aldehydes using NADH as cofactor as a result of overexpression of the SFA1 gene. Therefore a gene to be overexpressed for increasing the specific SFA1-encoded NADH-dependent alcohol dehydrogenase and glutathione-dependent aldehyde dehydrogenase activities in the cell of the invention, preferably is a gene encoding an amino acid sequence with at least 70, 75, 80, 85, 90, 95, 98 or 99% sequence identity to SEQ ID NO: 10.

In another embodiment, for increasing the resistance to acetaldehyde the cell of the invention comprises a (further) genetic modification that increases the intracellular glutathione (GSH) levels in the cell, as compared to a corresponding unmodified parent strain. The intracellular glutathione level in the cell is preferably increased by at least a factor 1.05, 1.1, 1.2, 1.5, 2.0, or 5.0 as compared to cells of a strain which is genetically identical except for the genetic modification causing the increased GSH level, at least when grown on glucose under anoxic conditions.

In yeast, GSH is synthesized in two consecutive ATP-dependent reactions. The first step, catalyzed by a γ-glutamylcysteine synthetase encoded by GSH1, has shown to be rate-limiting as overexpression of GSH2 led to unchanged levels of total glutathione, whereas overexpression of GSH1 resulted in an almost twofold increase in the intracellular GSH levels (Grant et al., 1997, Mol. Biol. Cell. 8:1699-1707). Increased expression of CYS3, encoding cystathionine-γ-lyase, was found in a UV-mutagenized strain of S. cerevisiae, which accumulated high levels of GSH (Nisamedtinov et al., 2011, Appl Microbiol Biotechnol, 89:1029-1037). Utilization of GSH results in the oxidation to its disulfide form, GSSG, from which GSH can be regenerated by the action of glutathione reductase, encoded by GLR1. Overexpression of glutathione reductase from Oryza sativa and Brassica rapa in S. cerevisiae has been shown to increase tolerance against oxidative stress (Kim et al., 2012, J Microbiol Biotechnol, 22:1557-1567; Yoon et al., 2012, World J Microbiol Biotechnol 2012, 28:1901-1915). Ask et al. (2013, Microbial Cell Factories 2013, 12:87) demonstrate that overexpression of at least the GSH1 gene, optionally in combination with the CYS3 and GLR1 genes can alleviate the toxic effects induced by lignocellulose derived inhibitors, which include aldehydes and thereby enhance robustness of S. cerevisiae for lignocellulosic hydrolysate fermentation.

Thus, a preferred gene to be overexpressed for increasing the intracellular GSH level in the cell of the invention is at least the S. cerevisiae GSH1 gene or an orthologue thereof from another species, preferably encoding an amino acid sequence with at least 70, 75, 80, 85, 90, 95, 98 or 99% sequence identity to SEQ ID NO: 11. In addition to GSH1 gene, further preferred genes to be overexpressed for increasing the intracellular GSH level in the cell of the invention are the S. cerevisiae CYS3 and GLR1 genes (with respectively Genbank accession no.'s BK006935.2 and NC_001148.4) or orthologues thereof from other species. The construction of yeast strains overexpressing one or more of the GSH1, CYS3 and GLR1 genes from expression constructs integrated in the yeast genome are described by Ask et al. (2013, supra).

In another embodiment, for increasing the resistance to acetaldehyde the cell of the invention comprises a (further) genetic modification that increases the intracellular lysine level in the cell, as compared to a corresponding unmodified parent strain. The intracellular lysine level in the cell is preferably increased by at least a factor 1.05, 1.1, 1.2, 1.5, 2.0, 5.0, 10, 20 or 50 as compared to cells of a strain which is genetically identical except for the genetic modification causing the increased lysine level, at least when grown on glucose under anoxic conditions.

Lysine residues in proteins have been implicated as target structures for acetaldehyde adducts and indeed Braun et al. (1995, J. Biol. Chem. 270:11263-66) have described that the amino terminal amine group, as well as the c-amine groups of the lysine side chain can serve as sites for modification by acetaldehyde. Lysine overproducing mutants of S. cerevisiae can be isolated by selecting for S. cerevisiae mutants resistant to toxic lysine analog S-2-aminoethyl-L-cysteine as described e.g. by Gasent-Ramirez and Benitez (1997, Appl. Environ. Microbiol. 63:4800-6). Lysine overproduction can also be achieved by the loss of repression of the homocitrate synthase encoded by the LYS20 repressor gene. Therefore, in a preferred yeast cell of the invention, the gene that is modified, by reducing or eliminating its expression for overproducing lysine, is the S. cerevisiae LYS20 gene, or an orthologue thereof in another species, which preferably is a gene encoding a amino acid sequence with at least 70, 75, 80, 85, 90, 95, 98 or 99% sequence identity to SEQ ID NO: 12.

3. Production of Acetaldehyde

The acetaldehyde to be used in a process of the invention can be produced in a variety of ways. For example, several processes exist for the chemical production of acetaldehyde. The economics of the different processes are strongly dependent on the price of the respective feedstocks used. Since 1960, the liquid-phase oxidation of ethylene has been the process of choice, although there also is commercial production by the partial oxidation of ethanol and by hydration of acetylene. Acetaldehyde is also formed as a co-product in the high temperature oxidation of butane. A recently developed rhodium-catalyzed process produces acetaldehyde from synthesis gas as a co-product with ethyl alcohol and acetic acid. Oxidation of ethanol is the oldest and the best laboratory method for preparing acetaldehyde. In the industrial version of this process, ethanol is oxidized catalytically with oxygen (or air) in the vapor phase, whereby copper, silver, and their oxides or alloys are usually used catalysts. One advantage of this process that it is a so-called green process, using renewable feedstocks.

Preferably therefore, in the process of the invention, the acetaldehyde to be applied in a process of the invention, is produced by catalytic oxidation of ethanol, preferably using a catalyst in the presence of oxygen, wherein the catalyst comprises a noble metal or an oxide thereof. Preferably, the noble metal is selected from silver, copper, platinum, gold and alloys thereof.

Eliasson (2010, http://www.chemeng.lth.se/exjobb/E572.pdf) discloses a design for a plant for manufacturing acetaldehyde from ethanol at a relatively small scale (16,000 tons per year). Using a silver catalyst for oxidation of the ethanol, the design achieves an overall yield for acetaldehyde from ethanol of 93%.

Advantageously, in one aspect of the invention, a facility for producing acetaldehyde by catalytic oxidation from ethanol, such as e.g. the one disclosed by Eliasson (2010, supra), can be integrated with an ethanol fermentation plant, thus substantially optimizing its overall economics.

Therefore, in a preferred embodiment, the production of the acetaldehyde to be applied in a process of the invention for producing ethanol is integrated with the process for producing ethanol. Preferably the two process are integrated in the sense that the acetaldehyde, to be applied in a process of the invention for producing ethanol, is produced, from a part of the ethanol obtained in step b) of a process for producing ethanol in accordance with the invention, preferably by catalytic oxidation of the ethanol as described herein above. More preferably, the two processes, i.e. the process for producing ethanol and the process for producing acetaldehyde, are integrated in a manner that minimizes transport (of ethanol and/or acetaldehyde) between the two types of processes. Preferably therefore, acetaldehyde production by catalytic oxidation of ethanol is performed at a site that is in the vicinity of (i.e. at a distance of less than 50, 20,10, 5, 2, 1, 0.5, 0.1, 0.05 or 0.01 km from) the site where the ethanol is produced. Most preferably, the acetaldehyde is produced “on-site” at the site where the ethanol is produced.

4. Ethanol Production System

In another aspect the invention pertains to system for producing ethanol. The system can be a plant or factory for producing ethanol. Preferably, the system is a system for producing ethanol in accordance with a process of the invention. The system therefore comprises at least a means for fermentation, preferably, a means fermentation of a medium to an ethanol-containing beer (fermented medium), preferably in accordance with a process of the invention. The system also preferably comprises a means for distillation, preferably, the means for distillation is for recovery of the ethanol from the beer. The means for distillation preferably produces a stream of ethanol. The system further preferably comprises a means for supplying acetaldehyde to the medium. A preferred system can further comprise a means for producing acetaldehyde by catalytic oxidation of ethanol.

The means for fermentation can comprise or consist of one or more fermenters or fermentation units. In the fermenter (or fermentation unit) a yeast cell ferments the medium containing or fed with a source of a fermentable carbohydrate and optionally a source of acetaldehyde to ethanol, preferably in accordance with a process of the invention. The means for fermentation can be means for performing a batch process, a fed-batch process or a multistage continuous fermentation process comprising several fermentation units, usually 2-4 units connected in series. Fermenters for yeast fermentation of carbohydrate to ethanol and carbon dioxide are well known in the art.

For recovery of the ethanol from the fermented medium or beer, the system preferably comprises a means for distillation. Thus, in the system of the invention, the ethanol-containing beer produced in the fermenter(s) is directed to a distillation unit. Here the beer is subjected to a distillation process where ethanol vapors are concentrated and separated from the beer/fermented medium. Various means and methods for distillation processes to produce high purity ethanol from fermented media are e.g. described by Madson (2003, supra).

The means for producing acetaldehyde by catalytic oxidation of ethanol can comprise or consist of at least a reactor comprising a catalyst that catalyses the oxidation of ethanol to acetaldehyde. The catalyst preferably as described hereinabove. The reactor comprising the catalyst preferably is fixed bed reactor over a bed of silver catalyst, e.g. a column. The means for producing acetaldehyde further preferably comprise a saturator, wherein air is saturated with ethanol, preferably pre-heated ethanol. The heat/energy generated by the exothermic catalytic oxidation may be used elsewhere in the system. e.g. in the means for distillation.

The system further, preferably, comprises, a means for supplying acetaldehyde to the fermentation medium. As described above, in a process of the invention, the acetaldehyde can be supplied to the fermentation medium in liquid and/or gaseous from. The acetaldehyde is preferably supplied to fermenter from a holding tank. The acetaldehyde supplied to fermenter can be obtained from the means for producing acetaldehyde by catalytic oxidation as described above, or from any other (external) source, including e.g. commercial supply of acetaldehyde.

When supplied in liquid form, the acetaldehyde can be pumped into the fermenter from a holding tank, comprising the acetaldehyde as such or in diluted form. Various means and methods are known in the art for supplying liquids to (fermentation) media that minimize the time of exposure of the organism to high concentrations of the supplied liquid.

Alternatively, the acetaldehyde is supplied to the fermenter in gaseous form. For supply in gaseous form the acetaldehyde is brought at a temperature above its boiling point (20° C. at atmospheric pressure) and then pumped or blown into the medium in the fermenter, whereby preferably the acetaldehyde is diluted/mixed with another gas.

Preferably, the gaseous acetaldehyde is mixed with carbon dioxide. More preferably, the acetaldehyde is diluted/mixed with at least a part of the (carbon dioxide-containing) off-gas stream from the fermenter. In this embodiment, the system is configured such that at least a part of the off-gas stream is recycled back into the fermenter after being mixed with gaseous acetaldehyde. Standard equipment for blowing gas into fermenters can be applied.

Preferably, the system comprises a means for detecting the acetaldehyde concentration, and optionally the ethanol concentration, in the fermentation medium. More preferably, the system comprises a means for on-line detection/monitoring of the acetaldehyde concentration and optionally the ethanol concentration, in the fermentation medium as described hereinabove, e.g. using a mass spectrometer or gas chromatography for on-line determination of acetaldehyde and ethanol concentrations in the gas (carbon dioxide) exhaust or off-gas streams from the fermenter.

Preferably, the system also comprises a means for controlling the rate of acetaldehyde supply into the medium in the fermenter. The means for controlling the rate of acetaldehyde supply into the medium preferably receives input from the means for monitoring/detecting the acetaldehyde concentration, to control/maintain an acetaldehyde concentration in the medium in accordance with a process of the invention. More preferably, the means for controlling the rate of acetaldehyde supply into the medium further receives input from the means for monitoring/detecting the ethanol concentration in the medium to further control the acetaldehyde concentration in the medium as a function of the ethanol concentration in accordance with a process of the invention. The means for controlling the rate of acetaldehyde supply into the medium in the fermenter can be configured to control the supply of acetaldehyde from a holding tank. Alternatively, the means for controlling the rate of acetaldehyde supply into the medium in the fermenter can be configured to control the supply of ethanol to the means for producing acetaldehyde by catalytic oxidation of ethanol, from which the acetaldehyde is fed into the medium in the fermenter, preferably without using a holding tank for the acetaldehyde.

A preferred system in accordance with the invention is a system essentially as described in the FIGURE.

In order to obtain >99% pure ethanol (by volume), that is required for some application, the ethanol can be further purified by a dehydration process. A typical dehydration process is performed using a molecular sieve as a desiccant as e.g. described by Bibb Swain (2003, supra). Preferably therefore, the system further comprises a molecular sieve system form absorbing water molecules from the process stream from the distillation unit.

The system may further comprise one or more of means for milling, liquefaction processing and/or holding of a feedstock comprising the source of fermentable carbohydrate and means for holding or storing the ethanol produced and denaturant.

5. A Process for Disinfecting a Fermenter

In one aspect, the invention relates to a process for disinfecting fermentation equipment. Preferably the process is a process for disinfecting a fermenter (i.e. a bioreactor).

Acetaldehyde is toxic to most microbes. For example, for Lactococcus lactis (Bongers et al. 2005. Applied and Environmental Microbiology 71:1109-1113), a minimum inhibitory concentration (MIC) of acetaldehyde of 35 mM (1.5 kg/m³) was detected whereas a concentration of 9 mM (0.4 kg/m³) resulted in a reduction of the growth rate by 50%. At the same time most microorganisms, including e.g. yeasts like S. cerevisiae, can tolerate lower concentrations of acetaldehyde and can metabolize acetaldehyde as it is already an intermediate in the natural metabolism in most microorganisms.

The process of the invention for disinfecting a fermenter, preferably comprises two steps. A first step wherein an amount of acetaldehyde is supplied to the fermenter, preferably prior to that the fermentation medium is introduced into the fermenter. The amount of acetaldehyde supplied to the fermenter preferably is such that it results in a concentration of acetaldehyde in the fermenter that will inactivate or kill most microbes. Preferably, the amount of acetaldehyde supplied to the fermenter results in a concentration of at least 1, 2, 5, 10, 20 or 50 kg/m³. In the first step of the method, the concentration of at least 1, 2, 5, 10, 20 or 50 kg/m³ is maintained for at least 1, 2, 5, 10, 20, 40 or 60 minutes. Preferably, at least a part of the acetaldehyde is introduced into the fermenter in the gas phase, and/or at least a part the acetaldehyde is brought into, and preferably kept in, the gas phase in the fermenter, e.g. by heating and/or reducing pressure in the fermenter.

Then, in a second step in the process of the invention for disinfecting a fermenter, fermentation medium, and optionally the fermentation organism, are introduced into the fermenter. The fermentation medium introduced into the fermenter preferably dilutes the acetaldehyde to a concentration that is no longer toxic to the fermentation organism.

In this aspect, a fermentation organism is understood as the organism that is to carry out a fermentation process subsequent to the disinfection process. A fermentation process can be any process in or on a medium (i.e. submerged or solid state fermentations) in a fermenter or bioreactor that involves the growth of the fermentation organism, the production of a fermentation product by the fermentation organism and/or a bioconversion performed by the fermentation organism. The fermentation organism can thus be any organism that can be used or cultured in fermentation processes, including e.g. plant cells, animal cells and microorganisms such as bacteria and fungi, including yeasts.

Preferably, in the process for disinfecting the fermenter, the amount of acetaldehyde that is introduced into the fermenter in the first step is chosen such that once the fermentation medium and, optionally also the fermentation organism, are introduced into the fermenter, the concentration acetaldehyde is reduced (diluted) to a concentration that does not significantly affect the performance of the fermentation organism. The performance of fermentation organism is understood as the growth- or production-rate of the organism and/or the rate of the bioconversion to be effected by the organism, which rates are preferably reduced to no less than 50, 60, 70, 80, 90, 95 or 98% of the rate achieved under identical conditions in the absence of added acetaldehyde. Preferably, therefore the supply of the medium and, optionally the fermentation organism, dilute the concentration of acetaldehyde in the fermenter to a concentration of no more than 2.0, 1.5, 1.0, 0.75, 0.50, 0.35, 0.25, 0.20, 0.15, 0.10, 0.08, 0.06, 0.05, 0.04, 0.02, 0.01 or 0.008 kg/m³.

In one embodiment, the process for disinfecting a fermenter, precedes a process for producing ethanol, preferably, the disinfection process precedes a process for producing ethanol in accordance with the invention. Preferably therefore, in a process for producing ethanol according to the invention, a process for disinfecting the fermenter is carried out prior to adding a least one of the medium and the yeast cell to the fermenter. Preferably, the process for disinfecting the fermenter preferably comprises two steps. A first step of: supplying to the fermenter an amount of acetaldehyde resulting in a concentration of acetaldehyde of at least 1, 2, 5, 10, 20 or 50 kg/m³, and, preferably incubating the acetaldehyde in the fermenter for at least 1, 2, 5, 10, 20, 40 or 60 minutes. Preferably, the amount of acetaldehyde supplied to the fermenter is such that upon subsequent supply of at least one of the medium and the yeast cell to the fermenter, the concentration of acetaldehyde is reduced (diluted) to a concentration that does not reduce the growth rate of the yeast cell to less than 50, 60, 70, 80, 90, 95 or 98% of the rate achieved under identical conditions in the absence of added acetaldehyde. Preferably, the amount of acetaldehyde supplied to the fermenter is such that upon subsequent supply of at least one of the medium and the yeast cell to the fermenter, the concentration of acetaldehyde is reduced (diluted) to a concentration of no more than 2.0, 1.5, 1.0, 0.75, 0.50, 0.35, 0.25, 0.20, 0.15, 0.10, 0.08, 0.06, 0.05, 0.04, 0.02 0.01 or 0.008 kg/m³. And a second step of: supplying medium and optionally yeast cells to the fermenter in an amount to dilute the acetaldehyde to a concentration of no more than 2.0, 1.5, 1.0, 0.75, 0.50, 0.35, 0.25, 0.20, 0.15, 0.10, 0.08, 0.06, 0.05, 0.04, 0.02 0.01 or 0.008 kg/m³. Preferably, at least a part of the acetaldehyde is introduced into the fermenter in the gas phase, and/or at least a part the acetaldehyde is brought into, and preferably kept in, the gas phase in the fermenter, e.g. by heating and/or reducing pressure in the fermenter.

In any of the processes of the invention, acetaldehyde can be used to prevent or reduced growth of microbial contaminants in a feedstock for the fermentation. Particularly during preparation and/or storage or holding of feedstock rich in carbohydrates or other carbon sources microbial infection can occur. To prevent or reduce microbial growth in such feedstocks, acetaldehyde is added to the feedstock in a concentration of at least 1, 2, 5, 10, 20 or 50 kg/m³. This concentration of acetaldehyde in the undiluted feedstock preferably is such that upon dilution of the feedstock into the fermentation medium, e.g. during its preparation or in a fed-batch process, the acetaldehyde is dilute in the fermentation medium to a concentration of no more than 2.0, 1.5, 1.0, 0.75, 0.50, 0.35, 0.25, 0.20, 0.15, 0.10, 0.08, 0.06, 0.05, 0.04, 0.02 0.01 or 0.008 kg/m³.

6. Use of Acetaldehyde

In yet another aspect, the invention relates to the use of acetaldehyde in a fermentation process for producing ethanol, preferably in a yeast fermentation process for producing ethanol. The acetaldehyde is used for at least one of: a) reducing the formation of glycerol; b) improving the performance of the yeast at high ethanol concentration; and, c) suppression of infection during the fermentation process. Preferably, the process for producing ethanol is a process of the invention as described herein above.

In yet another aspect, the invention relates to the use of acetaldehyde for disinfecting fermentation equipment such as e.g. a fermenter (i.e. a bioreactor). Preferably, the process for disinfecting fermentation equipment is a process of the invention as described herein above.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### DESCRIPTION OF THE FIGURE

A schematic illustration of an ethanol plant with a facility for catalytic oxidation of part of the ethanol produced in the plant to acetaldehyde and with a facility for supply of the acetaldehyde to the fermenter. In a fermenter (1) of e.g. 2000 m³, a medium comprising a fermentable carbohydrate and acetaldehyde is fermented with a yeast whereby the yeast cell ferments the fermentable carbohydrate and the acetaldehyde to ethanol. The ethanol-containing beer (9) is transferred to a distillation unit (2) for recovery of the ethanol (10), which is stored in an ethanol storage tank (3). Ethanol form the storage tank can be shipped for sales (11). A part (12) of the ethanol from the storage tank (3) is directed to a saturator (4), of e.g. 0.4 m³, wherein air (13) is saturated with ethanol. The air saturated with ethanol (14) is directed to a conversion column comprising e.g. a silver catalyst (5) of e.g. 0.8 m³, wherein the ethanol is oxidized to acetaldehyde (15), which can be held and optionally diluted with buffer in holding tank (6) of e.g. 10 m³. Heat/energy (16), that is generated by the exothermic catalytic oxidation in (5), can be used elsewhere in the plant, e.g. in the distillation unit (2). Diluted acetaldehyde (17) from the holding tank (6) is supplied to the fermenter (1) by dosage controller (7), which receives input (18) about the concentrations of acetaldehyde and ethanol in the fermentation medium, as detected by a detection unit (8) in the carbon dioxide off-gas stream (19) from the fermenter (1). The detection unit (8) can be a flame ionization detector.

### EXAMPLES

### Methods and Materials

### Strains

S. cerevisiae CEN.PK2-1C or similar CEN.PK strains are obtainable from EUROSCARF at the University of Frankfurt, Germany, e.g. via email Euroscarf@em.uni-frankfurt.de or at: http://web.uni-frankfurt.de/fb15/mikro/euroscarf/index.html.

S. cerevisiae CBS 8066 is obtainable from CBS-KNAW, Centraalbureau voor Schimmelcultures (CBS) Fungal Biodiversity Centre Centraal Bureau, Utrecht, the Netherlands, at www.cbs.knaw.nl.

The commercial yeast Thermosacc™ is obtainable from Lallemand Biofuels & Distilled Spirits, Duluth, Ga. 30097, USA (www.lallemandbds.com).

The commercial yeast Ethanol Red™ is obtainable from Phibro Animal Health Corporation, Ethanol Performance Group, Teaneck, N.J. 07666-6712, USA (www.ethanolperformancegroup.com).

The commercial yeast Fermiol Super HA Thermosacc™ is obtainable from Enzyme Development, New York.

### Metabolite Analyses

Supernatant obtained by centrifugation of culture samples was analyzed for glucose, ethanol, acetaldehyde, glycerol, acetic acid, succinic acid, pyruvic acid and 2,3-butanediol via HPLC analysis on a Waters Alliance 2690 HPLC (Waters, Milford, USA) containing a Biorad HPX 87H column (Biorad, Hercules, USA). The column was eluted at 60° C. with 0.5 g/l H₂S0₄ at a flow rate of 0.6 ml min⁻¹ . Detection was by means of a Waters 2410 refractive-index detector and a Waters 2487 UV detector. Initial and final glycerol concentrations were further determined using an enzymatic determination kit (Rbiopharm AG, Darmstadt, Germany).

For dry weight measurements nitrocellulose filters (pore size, 0.45 μm; Gelman Sciences, Inc., Ann Arbor, Mich.) were used. Samples were harvested at desired cultivation times. After removal of the medium by filtration, the filters were washed with demineralized water and dried in an R-7400 Microwave Oven (Sharp Inc., Osaka, Japan) for 15 min. This procedure yielded the same dry weight data as drying of filters at 80° C.

### Enzyme Activity Analyses

Cell extracts for activity assays were prepared and analyzed for protein content as described by Postma et al., (1989, Appl. Environ. Microbiol. 55(2):468).

Acetaldehyde dehydrogenases (NAD⁺ and NADP⁺) (EC 1.2.1.5 and EC 1.2.1.4, respectively) activity was measured at 30° C. by monitoring the oxidation of NADH or NADPH at 340 nm. The assay mixture contained potassium phosphate buffer (pH 8.0) (100 mM), pyrazole (15 mM), dithiothreitol (0.4 mM), KCl (10 mM), and NAD⁺ or NADP⁺ (0.4 mM). The reaction was started with 0.1 mM acetaldehyde.

For glycerol 3-phosphate dehydrogenase (EC 1.1.1.8) activity determination, cell extracts were prepared as described above except that the phosphate buffer was replaced by triethanolamine buffer (10 mM, pH 5). Glycerol-3-phosphate dehydrogenase activities were assayed in cell extracts at 30° C. as described previously (Blomberg and Adler, 1989, J. Bacteriol. 171: 1087-1092.9).

Glycerol 3-phosphatase was assayed as described previously (Norbeck et al. 1996. J. Biol. Chem. 271:13875-13881). Briefly, cell-free extracts were incubated in 20 mm Tricine-HCl (pH 6.5), 5 mM MgCl₂, and 10 mm dl-glycerol 3-phosphate in a total volume of 1.0 ml. After starting the reaction, samples of 90 μl were withdrawn at different time points and the reaction was stopped by adding 10 μl of 50% HClO₄. Inorganic phosphate was analyzed according to a previous study (27), and the reaction rate was calculated from the slope of a linear plot of released phosphate versus time. All glassware used was immersed overnight in 1 M HCl and rinsed thoroughly in distilled water, to eliminate phosphate contamination.

### Cultivation Procedures

The mineral salts medium employed was based on standard media (Bruinenberg et al. 1983. Journal of General Microbiology 129:965-971; Verduin et al 1990. Journal of General Microbiology 136:395-403). It contained the following per liter of demineralized water: (NH₄)₂SO₄, 5 g; KH₂PO₄, 3 g; MgSO₄.7H₂O, 0.5 g; EDTA, 15 mg; ZnSO₄.7H₂0, 4.5 mg; CoCl₂ 6H₂O, 0.3 mg; MnCl₂ 4H₂0, 1 mg; CuSO₄ 5H₂0, 0.3 mg; CaCl₂. 2H₂0, 4.5 mg; FeSO₄ 7H₂0, 3 mg; Na₂MoO₄. 2H₂O, 0.4 mg; H₃BO₃, 1 mg; KI, 0.1 mg; and 0.025 ml silicone antifoam (BDH). After heat sterilization at 120° C. and cooling, filter sterilized vitamins were added: biotin, 0.05 mg; calcium pantothenate, 1 mg; nicotinic acid, 1 mg; inositol, 25 mg; thiamin.HCl, 1 mg; pyridoxine.HCl, 1 mg; and para-aminobenzoic acid, 0.2 mg. Ergosterol and Tween 80 were dissolved in pure ethanol and steamed at 100° C. for 10 minutes before they were added to the medium to give final concentrations of 10 and 400 mg/l, respectively, and a final concentration of 38 mM ethanol. A glucose solution was heat-sterilized separately at 110° C. for 20 minutes and liquid acetaldehyde was used as such. The sterilized glucose solution was added to the sterile mineral salts medium to give the required final concentration.

Media for plates (1.5% agar) for S. cerevisiae were the mineral salts medium supplement with 2% glucose and for lactic acid bacteria the de Man, Rogosa and Sharpe (MRS) medium. Cycloheximide (0.1 g/l) was added to the plates in case mixed cultures of yeast and lactic acid bacteria were studied.

Small-scale batch cultivation of the yeast for testing the effect of acetaldehyde (1.7 mM) was done at 30° C. in capped bottles of 30 ml. Control bottles did not receive addition of acetaldehyde. Carbon dioxide was allowed to escape from the bottles by inserting a needle in the septum of the cap. The bottles were incubated stationary. A sample per bottle was taken at the start of the experiment and after 90 minutes of incubation.

Small-scale batch cultivation of the yeasts for optimizing acetaldehyde tolerance was done at 30° C. in 100-ml Erlenmeyer flasks. The flasks contained 20 ml mineral salts medium supplied with glucose (12 g/l) and they were incubated on a rotary shaker at 400 r.p.m. Air could enter in the flasks via cotton plugs.

Semi-anoxic batch cultivation of the yeasts together with lactic acid bacteria was done at 30° C. in 100-ml Erlenmeyer flasks. The flasks contained 50 ml mineral salts medium supplied with glucose (12 g/l) and they were incubated without shaking. Air could enter in the flasks via cotton plugs.

Anoxic chemostat cultivation of the yeasts was done at 30° C. in a fermenter with a working volume of 1 liter and at a stirring speed of 600 r.p.m. The pH was automatically controlled at pH=5 by titration with 2 M KOH. The condenser at the outlet of the gas stream was connected to a cryostat and cooled at 2° C. The condensate was returned into the fermentation vessel. The tubing on the entire fermenter set-up (including medium- and waste-reservoirs) consisted of material (Norprene tubing) that is very poorly permeable for oxygen. The fermenter and the medium reservoir (with a magnetic stirrer) were sparged with certificated ultra-pure nitrogen prior to the addition of acetaldehyde to the reservoir.

Batch cultivation for testing the effect of acetaldehyde on the production of glycerol and ethanol was done at 30° C. in a bioreactor with a working volume of 1 liter and a headspace of 0.5 liter. The stirring speed was set at 300 r.p.m. and the pH of the mineral salts medium containing 190 g/l glucose was kept at 5 by adding 2 M KOH. The condenser at the outlet of the gas stream was connected to a cryostat and cooled at 2° C. The condensate was returned into the fermentation vessel. The off-gas was monitored continuously with a Series 200ACE Acetaldehyde Analyzer (GOW-MAC Instrument Co). Sampling was from a loop kept at 35° C. for circulating gas from the headspace. Acetaldehyde was added by a peristaltic pump to the bioreactor intermittently as a solution of 25% acetaldehyde in water. The solution entered in the aqueous phase via a needle at the outlet of the tubing. The addition of the acetaldehyde solution was controlled and set as based on the measurements of the acetaldehyde concentration in the gas phase.

Batch cultivation for testing the effect of acetaldehyde at higher ethanol concentrations was done at 30° C. in a bioreactor with a working volume of 1 liters and a headspace of 0.5 liter. The stirring speed was set at 300 r.p.m. and the pH of the mineral salts medium containing 280 g/l glucose was kept at 5 by adding 2 M NaOH. The condenser at the outlet of the gas stream was connected to a cryostat and cooled at 2° C. The condensate was returned into the fermentation vessel. The off-gas was monitored continuously for both ethanol and acetaldehyde with a Series 200ACE Acetaldehyde Analyzer (GOW-MAC Instrument Co). Sampling was from a loop kept at 35° C. for circulating gas from the headspace. Acetaldehyde was added to the bioreactor once the ethanol concentration in the aqueous phase had reached 80 g/l as calculated from the concentrations in the gas phase. It was added by a peristaltic pump to the bioreactor intermittently as a solution of 10% acetaldehyde in water. The solution entered in the aqueous phase via a needle at the outlet of the tubing. The addition of the acetaldehyde solution was controlled and set as based on the measurements of the acetaldehyde concentration in the gas phase.

### Example 1

### Modification of Host Cells By Evolutionary Engineering

S. cerevisiae CEN.PK2-1C was subjected to evolutionary engineering with the aim of obtaining organisms that had acquired an enhanced tolerance to acetaldehyde. The organisms were cultivated under oxic conditions in a batch-wise mode. The initial pH of the mineral salts medium was set at 6 by titrating with KOH and glucose was added at 12 g/l. In the first round of incubations, acetaldehyde was added to this medium to reach a concentration of 0.2 g/l. After 3 days of incubation, an aliquot of 0.5 ml was taken from the culture and transferred into fresh medium (50 ml), now containing 0.3 g/l acetaldehyde. Subsequently, this procedure was repeated weekly with ever increasing acetaldehyde concentrations in the medium. The strains obtained in this way, had acquired increasing tolerance towards acetaldehyde. Strain S. cerevisiae CEN.PK2-1C now grew in the batch system in the presence of 0.5 g/l acetaldehyde without a noticeable change in growth rate as compared to the parent strain in the absence of acetaldehyde. The newly acquired strains retained their ability to withstand higher acetaldehyde levels also when cultivating them anoxically in mineral salts medium with glucose and for prolonged periods of times. This subculturing was by taking an aliquot of 0.5 ml from a culture that had consumed all glucose and by transferring this inoculum to fresh medium (50 ml). This procedure was repeated ten times before the stability towards acetaldehyde was assessed.

### Example 2

### Modification of the Host Cells Along with Lactic Acid Bacteria By Evolutionary Engineering

S. cerevisiae CEN.PK2-1C was subjected to evolutionary engineering analogous to the procedure described above, except that now semi-anoxic conditions were employed. Eight strains of lactic acid bacteria, belonging to Lactobacillus, Pediococcus, Leuconostoc and Weissella, were inoculated as well. The dynamics of the quantitative growth of the 9 organisms thus present as co-culture was followed during the procedure of evolutionary engineering. During each successive transfer step, the suspension after growth was plated on media that allow growth of either S. cerevisiae or of the 8 strains of lactic acid bacteria. In time, with increasing concentrations of acetaldehyde in the subsequent batches, the lactic acid bacteria disappeared from the incubations. Cultivation and subsequent transfers of the co-culture containing the 9 organisms was also done in the absence of acetaldehyde. Under this circumstance, the lactic acid bacteria remained present in the system.

### Example 3

### Anoxic Chemostat Fermentations Under Glucose Limitation in Either the Absence or Presence of Acetaldehyde

Strain S. cerevisiae CBS 8066 was cultivated in chemostat culture at a dilution rate of 0.11 h⁻¹. The glucose concentration in the medium reservoir was 24 g/l (137 mM) in each run. Three separate steady states were obtained in either the absence or presence of acetaldehyde. The effect of the aldehyde on the fermentation was tested by adding the compound to the medium reservoir after a steady state situation in its absence (medium and samples “1”) had been established. In a first acetaldehyde run, a concentration of 6 mM (0.26 g/l) acetaldehyde was included in the medium reservoir (medium and samples “2”). The second acetaldehyde run contained 15 mM (0.65 g/l) acetaldehyde in the reservoir (medium and samples “3”). In each of the three steady states (after 3-5 volume changes), the fermentation medium in the fermenter was analyzed for various compounds as summarized in Tables 1 and 2.

TABLE 1
Chemostat experiment: metabolite concentrations in the feed (medium) and in
duplicate (A and B) chemostat samples of the three steady states (1 = no acetaldehyde
supplied; 2 = 6 mM acetaldehyde supplied; and 3 = 15 mM acetaldehyde supplied).

| | | | | | | butane- | pyruvic | succinic |
|---|---|---|---|---|---|---|---|---|
| | glucose | acetaldehyde | glycerol | ethanol | acetic acid | diol | acid | acid |
| | (mM) | (mM) | (mM) | (mM) | (mM) | (mM) | (mM) | (mM) |
| Medium 1 | 137.78 | 0.00 | <1 | 37.86 | n.d. | n.d. | 2.13 | n.d. |
| Medium 2 | 136.00 | 6.00 | <1 | 38.27 | n.d. | n.d. | 2.14 | n.d. |
| Medium 3 | 136.27 | 14.79 | <1 | 38.49 | n.d. | n.d. | 2.12 | n.d. |
| Sample | 0.42 | 0.02 | 15.55 | 229.51 | 0.41 | n.d. | 0.54 | 0.45 |
| 1A | | | | | | | | |
| Sample | 0.44 | 0.03 | 15.34 | 227.99 | 0.28 | n.d. | 0.49 | 0.44 |
| 1B | | | | | | | | |
| Sample | 0.48 | 0.03 | 9.47 | 243.13 | 0.64 | n.d. | 0.56 | 0.43 |
| 2A | | | | | | | | |
| Sample | 0.46 | 0.03 | 9.60 | 244.74 | 0.54 | n.d. | 0.51 | 0.45 |
| 2B | | | | | | | | |
| Sample | 0.44 | 0.04 | 4.04 | 255.35 | 0.78 | n.d. | 0.48 | 0.45 |
| 3A | | | | | | | | |
| Sample | 0.48 | 0.04 | 4.02 | 255.25 | 0.69 | n.d. | 0.48 | 0.50 |
| 3B | | | | | | | | |

n.d. = not detected

TABLE 2
Effect of acetaldehyde additions on glycerol and ethanol production.

| | glucose | acetaldehyde | Δ glycerol | Δ ethanol |
|---|---|---|---|---|
| | consumed | consumed | produced | produced |
| | (mM) | (mM) | (mM) | (mM) |
| Steady state 1 | 138 | 0 | 0 | 0 |
| Steady state 2 | 136 | −6 | −6 | 15 |
| Steady state 3 | 136 | −16 | −11 | 27 |

### Example 4

### Batch Cultivation for Testing the Effect of Acetaldehyde

Strain S. cerevisiae CBS 8066 was employed in testing the effect of acetaldehyde on the formation of ethanol and glycerol from glucose at a concentration of 50 g glucose/l. The initial cell concentration was 0.45 g/l dry biomass. Acetaldehyde was supplied to the bottle in a single addition at the start of the incubation. In a separate control run, no acetaldehyde was supplied. The concentrations of acetaldehyde, glycerol and ethanol as determined at the start and at the end of the fermentation, is given in Table 3.

TABLE 3
Effect of acetaldehyde on the formation of ethanol and
of glycerol in a batch fermentation

| | mM | mM |
|---|---|---|
| Glucose at start | 280 | 280 |
| Glucose at end | Not determined | Not determined |
| Acetaldehyde in | — | 1.76 |
| Acetaldehyde at end | 0.12 | 1.01 |
| Ethanol at end | 6.39 | 6.67 |
| Glycerol at end | 1.07 | 0.58 |

### Example 5

### Effect of Acetaldehyde Additions on Batch Fermentations at High Ethanol Concentrations

The Ethanol Red strain was used in assessing the effect of acetaldehyde at high ethanol concentrations. The mineral salts medium was employed with the addition of yeast extract at 1 g/l. The initial cell concentration was 0.5 g/l dry biomass. Air was supplied to the headspace of the fermenter at 15 ml/min during the first 2 hours of cultivation after which conditions were kept anoxic. Glucose was added initially at 230 g/l. Acetaldehyde was added once the ethanol concentration in the fermentation had reached 80 g/l. The concentration was monitored from the headspace and controlled in the fermenter liquid between 0.06 and 0.1 g/l. In a control experiment, no acetaldehyde was supplied. The time required to reach 98 g/l of ethanol in the liquid as determined from its gas phase concentration was taken as criterion in assessing the effect of acetaldehyde. Without acetaldehyde it took 180 minutes to reach 98 g/l ethanol whereas in the presence of acetaldehyde this level had been reached in 158 minutes. The total amount of acetaldehyde supplied during the interval between 80 and 98 g/l ethanol was 0.7 g.

1. A process for producing ethanol comprising:
a) fermenting a medium with a yeast cell in a fermenter, whereby the medium contains or is fed with a source of a fermentable carbohydrate and with a source of acetaldehyde, and whereby the yeast cell ferments the fermentable carbohydrate and the acetaldehyde to ethanol; and,
b) recovery of the ethanol from the medium,
wherein the acetaldehyde is present in or fed into the medium at least during a stage in the process when the growth rate of the yeast cell is at least 0.005 h⁻¹.

2. The process of claim 1, wherein:
a) in a first phase of the process before a threshold ethanol concentration in the medium is reached, the rate of the acetaldehyde fed into the medium is controlled to maintain an acetaldehyde concentration of at least 0.0009 kg/m³ and, preferably no more than 1.0 kg/m³; and,
b) in a second phase of the process after the threshold ethanol concentration in the medium is reached, the rate of the acetaldehyde fed into the medium is controlled to maintain an acetaldehyde concentration of no more than 0.3 kg/m³, and preferably at least 0.01 kg/m³,
and wherein the threshold ethanol concentration is between 40 and 100 kg/m³.

3. The process of claim 2, wherein the acetaldehyde concentration in the medium is monitored on-line in an off-gas stream from the fermenter, preferably using a mass spectrometer or a gas chromatograph.

4. The process of claim 3, wherein the acetaldehyde concentration in the medium is monitored on-line in an off-gas stream from the fermenter using a mass spectrometer or a gas chromatograph.

5. The process of claim 1, wherein the acetaldehyde is fed into the medium in a liquid form or in gaseous form.

6. The process of claim 5, wherein the acetaldehyde that is fed into the medium in gaseous form is mixed with at least a part of the off-gas stream from the fermenter that is recycled back into the fermenter.

7. The process of claim 1, wherein the yeast cell is of a genus selected from the group consisting of Saccharomyces, Kazachstania and Naumovia.

8. The process of claim 7, wherein the yeast cell belongs to a species selected from the group consisting of Saccharomyces cerevisiae, S. bayanus, S. bulderi, S. cervazzii, S. cariocanus, S. castellii, S. dairenensis, S. exiguus, S. kluyveri, S. kudriazevii, S. mikatae, S. paradoxus, S. pastorianus, S. turicensis, and S. unisporus.

9. The process of claim 1, wherein the yeast cell ferments under anoxic conditions.

10. The process of claim 7, wherein the yeast cell has one or more modifications selected from the group consisting of:
a) a genetic modification that increases resistance to acetaldehyde as compared to a corresponding unmodified parent strain, whereby preferably the cell with increased resistance to acetaldehyde is obtained by one or more of:
i) evolutionary engineering;
ii) a genetic modification that increases specific NADH-dependent alcohol dehydrogenase activity, whereby preferably the alcohol dehydrogenase has an amino acid sequence with at least 70% sequence identity to SEQ ID NO: 9;
iii) a genetic modification that increases the specific NADH-dependent alcohol dehydrogenase and glutathione-dependent aldehyde dehydrogenase activities, whereby preferably the bifunctional NADH-dependent alcohol dehydrogenase and glutathione-dependent aldehyde dehydrogenase has an amino acid sequence with at least 70% sequence identity to SEQ ID NO: 10;
iv) a genetic modification that increases the intracellular glutathione level in the cell, whereby preferably, the genetic modification comprises at least the overexpression of a gene encoding a γ-glutamylcysteine synthetase, whereby preferably the γ-glutamylcysteine synthetase has an amino acid sequence with at least 70% sequence identity to SEQ ID NO: 11; and,
v) a genetic modification that increases the intracellular lysine level in the cell, whereby preferably the genetic modification confers resistance to S-2-aminoethyl-L-cysteine, or the genetic modification comprises reducing or eliminating the expression of a gene encoding a amino acid sequence with at least 70% sequence identity to SEQ ID NO: 12;
b) a genetic modification that reduces or eliminates endogenous aldehyde dehydrogenase activity, whereby preferably the genetic modification reduces or eliminates expression of endogenous S. cerevisiae ALD6 gene or an orthologue thereof;
c) a genetic modification that reduces or eliminates NADH-dependent glycerol synthesis, whereby preferably the genetic modification is a modification that reduces or eliminates the expression of one or more of the S. cerevisiae GPD1, GPD2, HOR2 and RHR2 genes or orthologues thereof;
d) a genetic modification that reduces or eliminates transport of glycerol, whereby preferably the genetic modification is a modification that reduces or eliminates the expression of the S. cerevisiae FPS1 gene or an orthologue thereof; and,
e) a genetic modification that introduces into the cell at least one of:
i) expression of an exogenous xylose isomerase gene, which gene confers to the cell the ability to isomerize xylose into xylulose; and,
ii) expression of exogenous genes coding for a L-arabinose isomerase, a L-ribulokinase and a L-ribulose-5-phosphate 4-epimerase, which genes together confer to the cell the ability to convert L-arabinose into D-xylulose 5-phosphate,
whereby the cell further preferably comprises genetic modifications that increase the specific activities of one or more of xylulose kinase, ribulose-5-phosphate isomerase, ribulose-5-phosphate 3-epimerase, transketolase and transaldolase; and a genetic modification that reduces or eliminates unspecific aldose reductase activity.

11. The process of claim 1, wherein the process for producing ethanol is preceded by a process for disinfecting the fermenter that is carried out prior to adding a least one of the medium and the yeast cell to the fermenter, wherein the process for disinfecting the fermenter comprises the steps of:
i) supplying to the fermenter an amount of acetaldehyde resulting in a concentration of acetaldehyde of at least 1 kg/m³, and incubating the acetaldehyde in the fermenter for at least 5 minutes, whereby the amount of acetaldehyde supplied is such that upon supply of the medium and the yeast cell to the fermenter, the concentration of acetaldehyde is diluted to no more than 2.0 kg/m³;
ii) supplying medium and optionally yeast cells to the fermenter in an amount to dilute the acetaldehyde to a concentration of no more than 2.0 kg/m³;
whereby, preferably, step i) the acetaldehyde is supplied into the fermenter in gas phase and/or the acetaldehyde is brought into the gas phase and or kept in the gas phase in the fermenter.

12. The process of claim 1, wherein the acetaldehyde is produced by catalytic oxidation of ethanol, preferably using a catalyst comprising one or more of a noble metal, an alloy thereof and oxides thereof, in the presence of oxygen, wherein preferably the noble metal is selected from silver, copper, platinum and gold.

13. The process of claim 12, wherein the acetaldehyde is produced by catalytic oxidation of a part of the ethanol obtained in b) of claim 1, whereby preferably the acetaldehyde is produced at a site in the vicinity of the site where the ethanol is produced.

14. A system for producing ethanol in a process according to claim 1, wherein the system comprises a means for fermentation of a medium to an ethanol-containing beer, a means for distillation for recovery of ethanol from the beer and a means for supplying acetaldehyde to the medium, and wherein, the system comprises a reactor holding a catalyst as defined in claim 8, for producing acetaldehyde by catalytic oxidation of ethanol.

15. The system of claim 14, wherein:
a) the system is configured to produce acetaldehyde by catalytic oxidation from a part of the ethanol obtained from the means for distillation, optionally after storage of the ethanol; and,
b) optionally, the system is configured to supply the acetaldehyde produced in a) to the medium, optionally after storage of the acetaldehyde.

16. The system of claim 14 or 15, wherein the system comprises a means for monitoring the acetaldehyde concentration and optionally the ethanol concentration, in the fermentation medium and a means for controlling the rate of the acetaldehyde supply into the medium in the fermenter, wherein preferably, the means for controlling the rate of acetaldehyde supply into the medium receives input from the means for monitoring the acetaldehyde concentration, and optionally the ethanol concentration, to maintain an acetaldehyde concentration in the medium in accordance with the process of claim 1, wherein preferably, the means for controlling the rate of acetaldehyde supply into the medium further receives input from the means for monitoring the ethanol concentration in the medium to further control the acetaldehyde concentration in the medium as a function of the ethanol concentration in accordance with the process of claim 2.

17. The system of claim 16, wherein the means for controlling the rate of acetaldehyde supply into the medium receives input from the means for monitoring the acetaldehyde concentration to control an acetaldehyde concentration in the medium in accordance with a process of claim 1, wherein preferably, the means for controlling the rate of acetaldehyde supply into the medium further receives input from the means for monitoring the ethanol concentration in the medium to further control the acetaldehyde concentration in the medium as a function of the ethanol concentration in accordance with the process of claim 2.
